(19) United States
(12) Patent Application Publication
(10) Pub. No.: US 2023/0256047 A1
(43) Pub. Date: Aug. 17, 2023

(54) METHODS AND COMPOSITIONS FOR THE TREATMENT OF MUSCULAR DYSTROPHY

(71) Applicant: STEALTH BIOTHERAPEUTICS INC., Needham, MA (US)

(72) Inventors: David BROWN, Needham, MA (US); Jim CARR, Needham, MA (US); Dennis Keefe, Needham, MA (US)

(73) Assignee: STEALTH BIOTHERAPEUTICS INC., Needham, MA (US)

(21) Appl. No.: 18/010,526

(22) PCT Filed: Jun. 16, 2021
(86) PCT No.: PCT/US2021/037581

§ 371 Date: Dec. 15, 2022

Related U.S. Application Data
(60) Provisional application No. 63/040,185, filed on Jun. 17, 2020.

(51) Int. Cl. A61K 38/07; A61P 9/00; A61P 21/00; A61K 31/56; A61K 31/7125; C12N 15/113

(52) U.S. Cl. CPC A61K 38/07; A61K 31/56; A61K 31/7125; A61P 9/00; A61P 21/00; C12N 15/113; C12N 2310/11

(57) ABSTRACT

The disclosure provides methods for treating muscular dystrophy (MD), such as Duchenne muscular dystrophy (DMD) or Becker muscular dystrophy (BMD) in a mammalian subject. The methods are particularly useful for treating, inhibiting, reducing, ameliorating or delaying the onset of hypertrophic cardiomyopathy, dilated cardiomyopathy, heart failure and/or cardiac fibrosis in subjects diagnosed with, and/or being treated for, MD.The methods comprise administering to the subject an effective amount of a peptide such as H-D-Arg-2,6-Dmt-Lys-PHe-NH₂ (a.k.a. elamipretide), or a pharmaceutically acceptable salt, hydrate, solvate and/or tautomer thereof, optionally in combination with at least one other active ingredient (i.e. drug) such a corticosteroid, ACE inhibitor, ARB(s), beta-blocker or drug that increases or corrects the expression of dystrophin in the subject (e.g. Eteplirsen (Exondys 51®), Golodirsen (Vyondys 53™)) or Casimersen (Amondys 45™).

### CROSS-REFERENCE TO RELATED APPLICATION

The present application claims priority to U.S. Provisional Pat. Application No. 63/040,185, filed on Jun. 17, 2020, the contents of which are hereby incorporated by reference in their entirety.

### TECHNICAL FIELD

The present disclosure relates generally to compositions (e.g. medicaments or formulations) methods and uses for treating heart disease (such as hypertrophic cardiomyopathy (HCM), dilated cardiomyopathy (DCM), heart failure and/or cardiac fibrosis) in subjects suffering from muscular dystrophy (MD, such as Duchenne muscular dystrophy (DMD) or Becker muscular dystrophy (BMD)) that result from a subjects impaired ability to produce the protein, dystrophin, and/or preventing, inhibiting, ameliorating or delaying the onset of such heart disease in said subjects. The present technology relates to administering, for example, an effective amount of a peptide and/or mixture of peptides such as H-D-Arg-2′6′-Dmt-Lys-Phe-NH₂ (more commonly known as elamipretide, SS-31, MTP-131 or bendavia); or a pharmaceutically acceptable salt, hydrate, solvate and/or tautomer thereof), and/or its carboxylate form, H-D-Arg-2′6′-Dmt-Lys-Phe-OH (or a pharmaceutically acceptable salt, hydrate, solvate and/or tautomer thereof), to a subject suffering from MD, DMD, or BMD, such as those at risk for developing, or who have developed, heart disease. Such treatments are particularly relevant to, and may be synergistic with, treatments involving the co-administration of corticosteroid(s), ACE inhibitor(s), ARB(s), beta-blocker(s) or a drug/medication that increases the production of dystrophin in muscle (for example, those subjects being treated with a phosphorodiamidate morpholino oligomer (PMO), such as Exondys 51® (Eteplirsen), Vyondys 53™ (Golodirsen) or Amondys 45™ (Casimersen)), or with a PPMO (defined below).

### INTRODUCTION

The following description is provided to assist the understanding of the reader. None of the information provided or references cited is admitted as prior art to the compositions and methods disclosed herein.

Muscular dystrophy (MD) is a group of inherited non-inflammatory but progressive muscle disorders. Duchenne muscular dystrophy (DMD) is the most common muscular dystrophy affecting 1 in about 3500 males born worldwide. Becker muscular dystrophy (BMD) is milder than DMD and primarily causes heart problems. BMD affects only males (1 in about 30,000), usually first appears between the ages of 2 and 16 years but can appear as late as age 25. Both DMD and BMD result from abnormal or deficient production of the protein, dystrophin.

DMD begins with progressive muscle weakness that evolves to loss of ambulation and further progresses to early morbidity and mortality. DMD is caused by mutations in the dystrophin gene at locus Xp21, located on the short arm of the X chromosome. Dystrophin encodes a 427-kD protein that plays an integral role in the structural stability of the myofiber. The loss of dystrophin disrupts the muscle membrane and fibers. Without dystrophin, muscle fibers are susceptible to mechanical injury and necrotic/apoptotic cell death.

DMD is a progressive disease which eventually affects all voluntary muscles as well as cardiac and breathing muscles in later stages. The disease is most prevalent in males. While female carriers of the DMD mutation are largely asymptomatic, some (20-30%) present with mild to moderate muscle weakness and are at increased risk for developing DCM. Boys generally present with symptoms between the ages of three to five years. These symptoms generally worsen over time leading to loss of ambulation and the need for a wheelchair by early adolescence. Further progression of DMD leads to respiratory distress and cardiomyopathies, which is present in almost all males by the age of 18. The average life expectancy for individuals afflicted with DMD is around age 25.

Signs and symptoms of DMD include progressive proximal weakness with onset in the legs and pelvis, hyperlordosis with wide-based gait, hypertrophy of weak muscles, pseudohypertrophy (enlargement of calf and deltoid muscles with fat and fibrotic tissue), reduced muscle contractility on electrical stimulation in advanced stages of the disease, delayed motor milestones, progressive inability to ambulate, heel cord contractures, paralysis, fatigue, skeletal deformities including scoliosis, muscle fiber deformities, cardiomyopathy, congestive heart failure or arrhythmia, muscular atrophy, respiratory disorders, bladder or bowel dysfunction, sensory disturbance, or febrile illness. Weakness of skeletal muscle can contribute to cardiopulmonary complications. Scoliotic deformity from paraspinal muscle asymmetric atrophy can impair pulmonary and gastrointestinal function, predisposing individuals to pneumonia, respiratory failure, and poor nutrition. Smooth muscle dysfunction as a result of abnormal or absent dystrophin, along with inactivity, leads to gastrointestinal dysmotility, causing constipation and diarrhea.

DMD can be diagnosed in several ways. A clinical diagnosis may be made when a male child has progressive symmetrical muscle weakness. Muscle biopsy is an important tool for quantifying the amount of muscle dystrophin as well as for detecting asymptomatic female carriers of DMD. Immunostaining of the muscle using antibodies directed against the rod domain, carboxy-terminals, and amino-terminals of dystrophin protein shows absence of the usual sarcolemma staining in boys with DMD. A combination of clinical findings, family history, blood concentration of creatine phosphokinase and muscle biopsy with dystrophin studies confirms the diagnosis (Creatine phosphokinase is normally present in high concentrations in the muscle cells). However, DMD patients exhibit creatine phosphokinase levels that are 50-100 times the reference range (as high as 20,000 mU/mL) during the early stages of the disease). Electromyography, electrocardiogram and echocardiogram, and lung monitoring tests may be used for confirmatory diagnosis of DMD. The progression of DMD occurs in 5 stages: presymptomatic, early ambulatory, late ambulatory, early nonambulatory, and late nonambulatory.

As with other aspects of DMD, the cardiomyopathies are progressive but generally end with heart failure. Ultrasonography can detect structural changes in the myocardium well before the onset of systolic dysfunction and overt cardiomyopathy. Despite the high incidence of heart failure, the majority of children with DMD are relatively asymptomatic until late in the disease course, probably because of their inability to exercise. Heart failure and arrhythmias may develop in the late stages of the disease, especially during intercurrent infections or surgery. The late-stage cardiomyopathy is characterized by extensive fibrosis of the posterobasal left ventricular wall followed by spread of the fibrosis to the lateral free wall of the left ventricle. The continued progression of the cardiomyopathy often leads to output failure and pulmonary congestion. Alternatively, cardiac fibrosis can include cardiomyopathy and conduction abnormalities, which can induce fatal arrhythmias. Heart failure is the most common cause of death of persons afflicted with DMD.

Myocardial energy homeostasis is disrupted in DMD, with dysfunctional mitochondria being a central factor. Impaired mitochondrial function in the dystrophic heart is observed early in both animal DMD models and human studies, often before observable declines in cardiac function. The lack of dystrophin leads to cellular membrane fragility and heightened susceptibility to membrane rupture.

The loss of cell membrane integrity induces ‘leaky’ fluxes of ions, enzymes, and metabolites. The influx of calcium is particularly problematic in the heart, which requires tightly regulated calcium cycling for pump function. Calcium content is normally three to four orders of magnitude lower in the cytosol compared to the outside of the cell. Heightened calcium within DMD myocytes causes sarcomeric disruption and calcium overload in mitochondria.

Mitochondrial calcium overload leads to several inter-related problems in the DMD heart. Calcium overload opens the mitochondrial permeability transition pore, a non-specific mitochondrial channel that can initiate apoptotic cell death. Opening of this pore can be catastrophic for mitochondria, as it collapses electrochemical and metabolite gradients that are crucial for ATP generation. DMD mitochondria have heightened production of reactive oxygen species, which can exacerbate cellular damage. Ruptured mitochondrial fragments can leak out of cells and contribute to inflammatory signaling cascades. Finally, mitochondrial structure, which is directly related to bioenergetic function, is compromised in DMD.

Mitochondrial dysfunction in DMD is a key contributor to cellular death. As the regenerative capacity of the heart is very low, the loss of myocytes places an increased burden on the surviving cells. Mitochondria within viable cells are under heightened pressure to meet the constant ATP demands of the heart. Futile pathological cycles continue to overwhelm cellular defense mechanisms as the disease progresses. Ensuing cardiac remodeling leads to higher propensity for electromechanical dysfunction and ultimately compromised cardiac function.

Historically, DMD and BMD patients have been treated for their symptoms. The standard of care has been treatment with corticosteroids to increase muscle function, ACE inhibitors, ARBs and beta blockers to address the progressive cardiomyopathy and assistive devices to address ambulatory needs. More recently, drugs based on an exon skipping mechanism of action that are directed to upregulation in the expression of the protein, dystrophin, in DMD patients (i.e. Exondys 51® (Eteplirsen), Vyondys 53™ (Golodirsen) or Amondys 45™ (Casimersen)) has been approved by the United States Food and Drug Administration (FDA). While these drugs appear to increase the expression of dystrophin in skeletal muscle, they do not appear to improve dystrophin expression in heart muscle. Indeed there does not appear to be any evidence that these drugs slow the progression of cardiomyopathies in DMD patients and indeed may hasten the progression of heart disease and the related cardiomyopathies if the increases in skeletal muscle function lead to increasing strength and physical exertion of the DMD patient which thereby increases the stress put on the patient’s heart resulting from increased physical activity.

In summary, although there are current treatments for symptoms and recent advances in medicine have begun to address the molecular underpinnings of the disease, MD (including without limitation DMD and BMD) remains an incurable illness for which additional treatments and therapies are desperately needed.

### SUMMARY

In one aspect, the present disclosure provides a method for treating cardiomyopathy or delaying the onset of cardiomyopathy in a mammalian subject suffering from muscular dystrophy, comprising administering to the subject a therapeutically effective amount of a peptide of formula A:

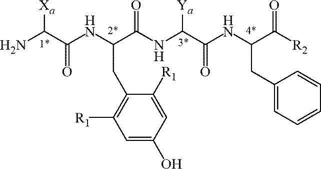

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

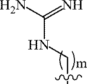

and

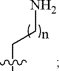

each m is 2, 3 or 4; each n is independently 1, 2, or 3; and the absolute stereochemistry at each of stereocenters 1^{∗}, 2^{∗}, 3^{∗}, and 4^{∗} is independently D or L.

In some embodiments, the peptide of generic Formula A is a peptide of formula A-1:

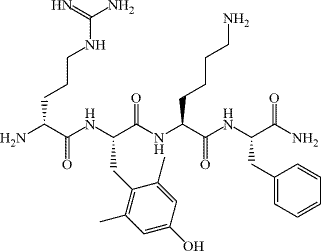 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

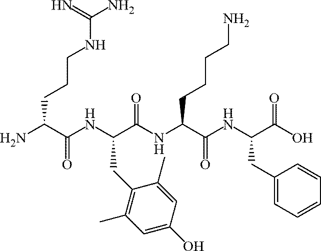 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-3, A-4, A-5, A-6, A-7 or A-8:

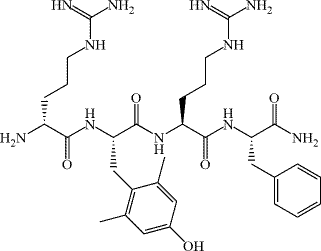 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O]

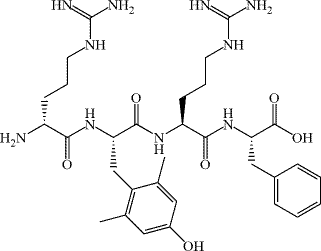 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O]

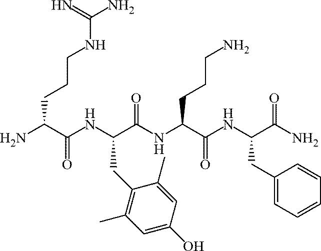 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O]

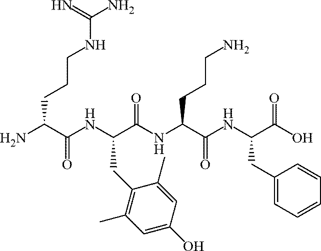 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

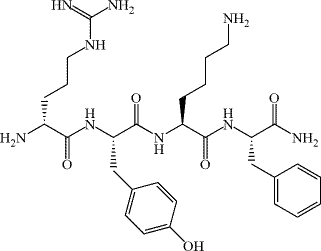 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O]

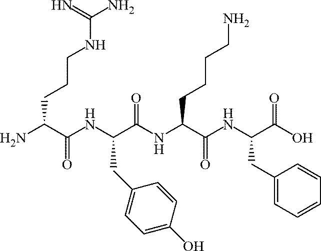 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate, and/or tautomer thereof.

In some embodiments, administration of the peptide reduces, ameliorates, and/or delays the onset of hypertrophic cardiomyopathy, dilated cardiomyopathy, heart failure and/or cardiac fibrosis in a subject diagnosed with and/or being treated for muscular dystrophy.

In some embodiments, administration of the peptide prevents, inhibits, reduces, ameliorates, and/or delays the onset of hypertrophic cardiomyopathy.

In some embodiments, administration of the peptide prevents, inhibits, reduces, ameliorates, and/or delays the onset of dilated cardiomyopathy.

In some embodiments, administration of the peptide prevents, inhibits, reduces, ameliorates, and/or delays the onset of heart failure.

In some embodiments, administration of the peptide prevents, inhibits, reduces, ameliorates, and/or delays the onset of cardiac fibrosis.

In some embodiments, administration of the peptide increases the ejection fraction, shortening fraction, stroke volume, or cardiac output of the heart of the subject as compared with the heart of an untreated control subject or control group that is not administered the peptide.

In some embodiments, the peptide is administered daily for: (i) 24 weeks or more; (ii) 48 weeks or more; (iii) 72 weeks or more; or (iv) 96 weeks or more. In some embodiments, the peptide is administered weekly for: (i) 24 weeks or more; (ii) 48 weeks or more; (iii) 72 weeks or more; or (iv) 96 weeks or more. In some embodiments, the peptide is administered daily or weekly to the subject from (or near to) the time of diagnosis until the end of life or near the end of life.

In some embodiments, the peptide is administered orally, topically, systemically, intraperitoneally, intradermally, transdermally, ophthalmically, intrathecally, intracerebroventricularly, iontophoretically, transmucosally, intravitreally, intranasally, or intramuscularly. In some embodiments, the peptide is administered subcutaneously or intravenously. In some embodiments, the subject is human.

In some embodiments, the muscular dystrophy is Duchenne muscular dystrophy (DMD). In some embodiments, the muscular dystrophy is Becker muscular dystrophy (BMD).

In some embodiments, the method further comprises separately, sequentially, or simultaneously administering an additional therapeutic agent to the subject.

In some embodiments, the peptide is administered to the subject in combination with a drug known to increase or correct the production of dystrophin in the subject. In some embodiments, the subject has been diagnosed as having DMD and the peptide is administered to the subject in combination with a phosphorodiamidate morpholino oligomer (PMO) such as Eteplirsen (Exondys 51®), Golodirsen (Vyondys 53™) or Casimersen (Amondys 45™) or a PPMO as the drug known to increase or correct the production of dystrophin in the subject.

In some embodiments, the peptide and the PMO or PPMO are administered intravenously. In some embodiments, the peptide and the PMO or PPMO are administered simultaneously.

In some embodiments, the peptide is administered to the subject in combination with a corticosteroid. In some embodiments, the peptide is administered to the subject in combination with an ACE inhibitor. In some embodiments, the peptide is administered to the subject in combination with an ARB. In some embodiments, the peptide is administered to the subject in combination with a beta blocker.

In some embodiments, the combined administration of peptide and additional therapeutic agent has a synergistic effect in the treatment of DMD or BMD.

In some embodiments, the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. Other pharmaceutically acceptable salts may also be used; many nonlimiting examples of which are provided in the definition of “pharmaceutically acceptable salt” or otherwise described in more detail below.

In some embodiments, the peptide of Formula A is administered in a depot formulation. In some embodiments, the depot formulation comprises the peptide of Formula A encapsulated or otherwise disposed in silica microparticles. In some embodiments, the depot formulation is a sustained release depot formulation. In some embodiments, the peptide of Formula A is released in an effective amount over days, weeks or months.

In one aspect, the present disclosure relates to the use of a composition in the preparation of a medicament for treating cardiomyopathy or delaying the onset of cardiomyopathy in a mammalian subject suffering from muscular dystrophy, wherein the composition comprises a therapeutically effective amount of a peptide of formula A:

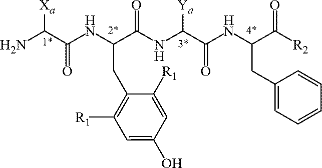

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

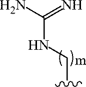

and

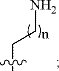

each m is 2, 3 or 4; each n is independently 1, 2, or 3; and the absolute stereochemistry at each of stereocenters 1^{∗}, 2^{∗}, 3^{∗}, and 4^{∗} is independently D or L.

In some embodiments, the peptide of generic Formula A is a peptide of formula A-1:

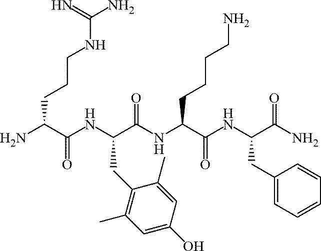 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

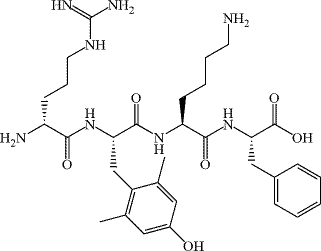 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-3, A-4, A-5, A-6, A-7 or A-8:

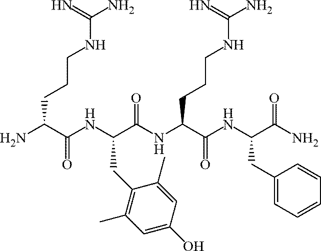 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O]

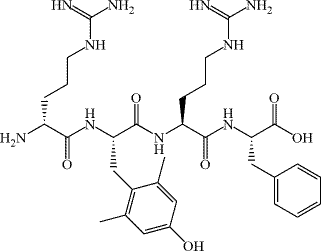 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O]

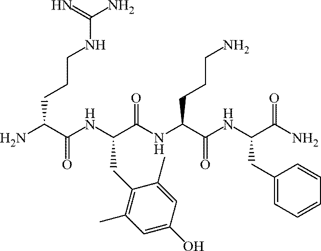 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O]

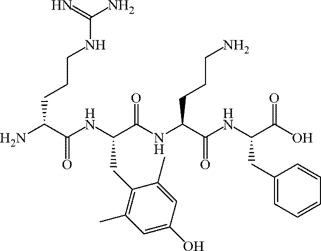 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

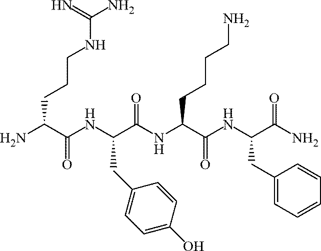 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O]

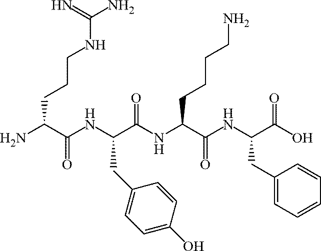 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the medicament further comprises a drug known to increase or correct the production of dystrophin in the subject. In some embodiments, the subject has been diagnosed as having DMD and the drug known to increase or correct the production of dystrophin is phosphorodiamidate morpholino oligomer (PMO), such as Eteplirsen (Exondys 51®), Golodirsen (Vyondys 53™) or Casimersen (Amondys 45™), or a PPMO.

In some embodiments, the muscular dystrophy is Duchene muscular dystrophy. In some embodiments, the muscular dystrophy is Becker muscular dystrophy.

In some embodiments, the cardiomyopathy is hypertrophic cardiomyopathy. In some embodiments, the cardiomyopathy is dilated cardiomyopathy. In some embodiments, the cardiomyopathy is heart failure. In some embodiments, the cardiomyopathy is cardiac fibrosis.

In some embodiments, the medicament increases ejection fraction of the heart of the subject as compared with the heart of an untreated control subject or control group that is not administered the composition.

In some embodiments, the medicament increases shortening fraction of the heart of the subject as compared with the heart of an untreated control subject or control group that is not administered the composition.

In some embodiments, the medicament increases stroke volume of the heart of the subject as compared with the heart of an untreated control subject or control group that is not administered the composition.

In some embodiments, the medicament increases cardiac output of the heart of the subject as compared with the heart of an untreated control subject or control group that is not administered the composition.

In some embodiments, the medicament decreases or delays the onset of cardiac fibrosis of the heart of the subject as compared with the heart of an untreated control subject or control group that is not administered the composition.

In one aspect, the present disclosure provides a composition comprising:
a) a peptide of formula A:

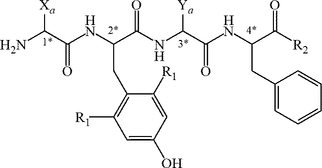

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

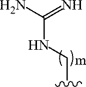

and

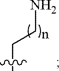

each m is 2, 3 or 4; each n is independently 1, 2, or 3; and the absolute stereochemistry at each of stereocenters 1^{∗}, 2^{∗}, 3^{∗}, and 4^{∗} is independently D or L; and
b) a drug known to increase or correct the production of dystrophin in a subject.

In some embodiments, the peptide of generic Formula A is a peptide of formula A-1 or A-2:

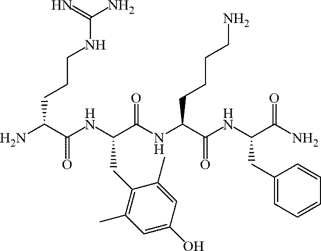 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

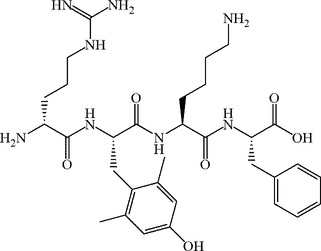 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

In some embodiments, the drug known to increase or correct the production of dystrophin is a phosphorodiamidate morpholino oligomer (PMO), such as Eteplirsen (Exondys 51®), Golodirsen (Vyondys 53™) or Casimersen (Amondys 45™), or a PPMO.

In some embodiments, the composition is a medicament.

In some embodiments, the present disclosure provides a method for treating cardiomyopathy or delaying the onset of cardiomyopathy in a mammalian subject suffering from muscular dystrophy, comprising administering to the subject a therapeutically effective amount of the composition.

In some embodiments, the composition is administered daily, weekly or monthly.

In some embodiments, the composition is administered intravenously.

In some embodiments, the muscular dystrophy is Duchene muscular dystrophy or Becker muscular dystrophy.

In one aspect, the present disclosure provides formulation comprising a peptide of formula A:

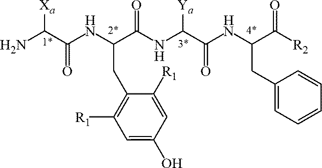

or a pharmaceutically acceptable salt, hydrate, solvate, and/or tautomer thereof, wherein, each R₁ is independently H or —CH₃; R₂ is —OH or —NH₂; Xₐ and Yₐ are each independently selected from

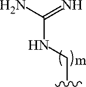

and

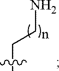

each m is 2, 3 or 4; each n is independently 1, 2, or 3; and the absolute stereochemistry at each of stereocenters 1^{∗}, 2^{∗}, 3^{∗}, and 4^{∗} is independently D or L, wherein: (i) said peptide is encapsulated by, or disposed within, silica microparticles; and (ii) said silica microparticles are formulated for systemic delivery of the peptide to a subject over days, weeks or months to thereby deliver an effective dose to thereby treat the subject for one or more signs, symptoms, or risk factors of cardiomyopathy associated with MD, DMD, or BMD.

### BRIEF DESCRIPTION OF THE DRAWINGS

FIG. 1 is an illustration of a peptide tetramer compound of general Formula A and two exemplary peptides A-1 (H-D-Arg-2,6-Dmt-Lys-Phe-NH₂) and A-2 (H-D-Arg-2,6-Dmt-Lys-Phe-OH).

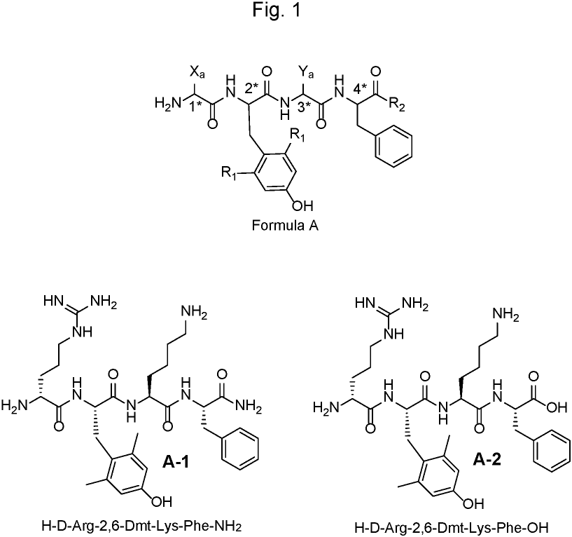

FIG. 2 is an illustration of various salt forms of the tetrapeptide of exemplary peptide A-2.

FIG. 3 is an illustration of various salt forms of the tetrapeptide of exemplary peptide A-1 (elamipretide).

### DEFINITIONS

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended claims, the singular forms “a”, “an” and “the” include plural references unless the content clearly dictates otherwise. For example, reference to “a cell” includes a combination of two or more cells, and the like.

As used herein, the “administering” or the “administration” of an agent (e.g. a peptide) or drug to a subject refers to any route of introducing or delivering to a subject a compound (e.g. peptide or mixture of peptides) to perform its intended function. Administration can be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or the administration by a device (e.g. a pump).

As used herein, the term “amino acid” refers to naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein the term or phrase “carrier” and “pharmaceutically acceptable carrier” refer to a diluent, adjuvant, excipient, or vehicle with which a peptide/compound/composition is administered or formulated for administration. Nonlimiting examples of such pharmaceutically acceptable carriers include liquids, such as water, saline, and oils; and solids, such as gum acacia, gelatin, starch paste, talc, keratin, colloidal silica, silica particles (nanoparticles or microparticles), urea, and the like. In addition, auxiliary, stabilizing, thickening, lubricating, flavoring, and coloring agents may be used. Other examples of suitable pharmaceutical carriers are described in Remington’s Pharmaceutical Sciences by E.W. Martin, herein incorporated by reference in its entirety.

As used herein, the phrase “delaying the onset of” refers to, in a statistical sample, postponing, hindering, or causing one or more symptoms of a disorder, symptom, condition or indication to occur more slowly than normal in a treated sample relative to an untreated control sample.

As used herein, the term “effective amount” refers to a quantity sufficient to achieve a desired therapeutic and/or prophylactic effect, e.g., an amount that treats, inhibits, reduces, ameliorates, or delays the onset of a cardiomyopathy or heart failure. In the context of therapeutic or prophylactic applications, in some embodiments, the amount of a composition administered to the subject will depend on the type and severity of the disease and on the characteristics of the individual, such as general health, age, sex, body weight and tolerance to drugs. The skilled artisan will be able to determine appropriate dosages depending on these and other factors. The peptide(s)/compound(s)/composition(s) disclosed herein can be administered in an effective amount prior to the onset of a cardiomyopathy associated with MD or in response to a cardiomyopathy that occurs in a subject suffering from MD. The peptide(s)/compound(s)/composition(s) disclosed herein can also be administered in combination with one or more additional therapeutic compounds (a so called “co-administration” where, for example, the additional therapeutic compounds could be administered simultaneously, sequentially or by separate administration). The one or more additional therapeutic compounds/compositions could be, for example, a corticosteroid, an ACE inhibitor, an ARB, a beta-blocker and/or a drug known to increase or correct the production of dystrophin in a subject (e.g. a phosphorodiamidate morpholino oligomer (PMO) such as Eteplirsen (Exondys 51®), Golodirsen (Vyondys 53™) or Casimersen (Amondys 45™) or a PPMO). In some embodiments, the co-administration of a peptide (or mixture of peptides) may produce a synergistic therapeutic effect.

In the methods described herein, therapeutic compounds (e.g. a peptide or mixture of peptides), or pharmaceutically acceptable salts, stereoisomers, mixtures of stereoisomers, tautomers, hydrates, and/or solvates thereof, may be administered to a subject having one or more signs, symptoms, or risk factors of cardiomyopathy associated with MD, DMD, or BMD. For example, a “therapeutically effective amount” of therapeutic compound (e.g. a peptide or mixture of peptides) includes levels at which the presence, frequency, or severity of one or more signs, symptoms, or risk factors of the cardiomyopathy are inhibited, reduced or eliminated. In some embodiments, a therapeutically effective amount reduces or ameliorates the physiological effects of a hypertrophic cardiomyopathy, dilated cardiomyopathy, heart failure and/or cardiac fibrosis.

As used herein, the term “hydrate” refers to a compound (e.g. a peptide or mixture of peptides) which is associated with water. The number of the water molecules contained in a hydrate of a compound may be (or may not be) in a definite ratio to the number of the compound molecules in the hydrate.

As used herein, the term “inhibit” “inhibits” or “inhibiting” means to reduce by an objectively measurable amount or degree compared to control. In one embodiment, inhibit or inhibiting means reduce by at least a statistically significant amount compared to control. In some embodiments, inhibit or inhibiting means reducing by at least 1-5 percent compared to control. In various individual embodiments, inhibit or inhibiting means reducing by at least 2, 3, 4, 5, 6, 7, 8, 9, 10, 15, 20, 25, 30, 33, 40, 50, 60, 67, 70, 75, 80, 90, 95, or 99 percent compared to control.

As used herein, the term “separate” with respect to a therapeutic use refers to an administration of at least two active ingredients at the same time or at substantially the same time by different routes. The “active ingredients” can, for example, be a peptide or mixture of peptides as disclosed herein and at least one of a corticosteroid, an ACE inhibitor, an ARB, a beta-blocker or a drug known to increase or correct the production of dystrophin in a subject (e.g. a phosphorodiamidate morpholino oligomer (PMO) such as Eteplirsen (Exondys 51®), Golodirsen (Vyondys 53™) or Casimersen (Amondys 45™) or a PPMO).

As used herein, the term “sequential” with respect to a therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition. The “active ingredients” can, for example, be a peptide or mixture of peptides as disclosed herein and at least one of a corticosteroid, an ACE inhibitor, a beta-blocker or a drug known to increase or correct the production of dystrophin in a subject (e.g. a phosphorodiamidate morpholino oligomer (PMO) such as Eteplirsen (Exondys 51®), Golodirsen (Vyondys 53™) or Casimersen (Amondys 45™) or PPMO).

As used herein, the term “simultaneous” with respect to a therapeutic use refers to the administration of at least two active ingredients (i.e. two pharmaceutically active ingredients) by the same or different route but at the same time or at substantially the same time. The “active ingredients” can, for example, be a peptide or mixture of peptides as disclosed herein and at least one of a corticosteroid, an ACE inhibitor, an ARB, a beta-blocker or a drug known to increase or correct the production of dystrophin in a subject (e.g. a phosphorodiamidate morpholino oligomer (PMO) such as Eteplirsen (Exondys 51®), Golodirsen (Vyondys 53™) or Casimersen (Amondys 45™) or a PPMO).

As used herein, the term “subject” refers to a living animal. In various embodiments, a subject is a mammal. In some embodiments, a subject is a non-human mammal, including, without limitation, a mouse, rat, hamster, guinea pig, rabbit, sheep, goat, cat, dog, pig, minipig, horse, cow, or non-human primate. In some embodiments, the subject is a human.

As used herein, the term “treat”, “treats”, “treating” or “treatment” refers to therapeutic treatment, wherein the object is to reduce, alleviate or delay onset of the progression or advancement of, and/or reverse the progression of the targeted pathological condition or disorder.

As used herein, “peptide-conjugated PMOs (PPMOs)” are PMOs to which a cell penetrating peptide is linked in order to improve cellular uptake of the PMO. See: Tsoumpra et al. (2019) “Peptide-conjugated antisense based splice-correction for Duchenne muscular dystrophy and other neuromuscular diseases” EBioMedicine, 45, 630-645; doi.org/10. 1016/j.ebiom.2019.06.036.

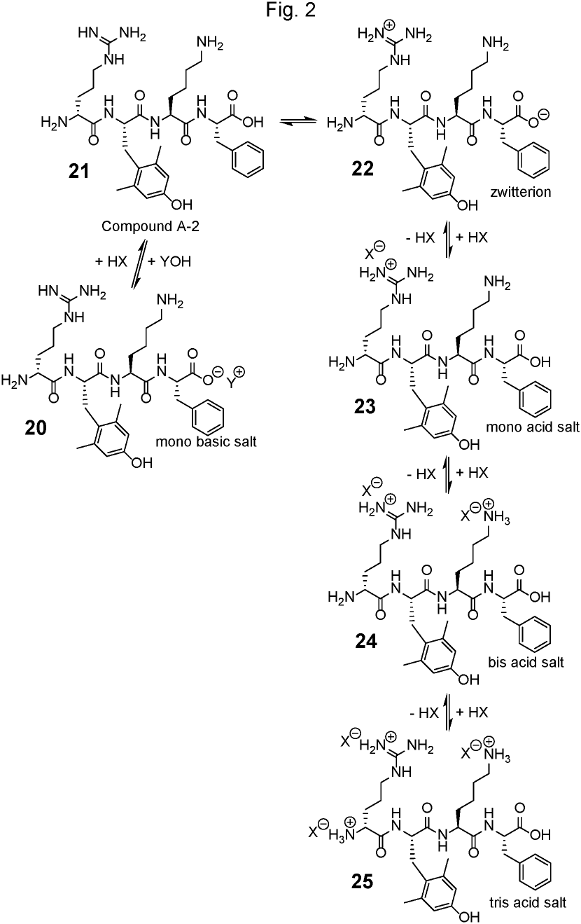

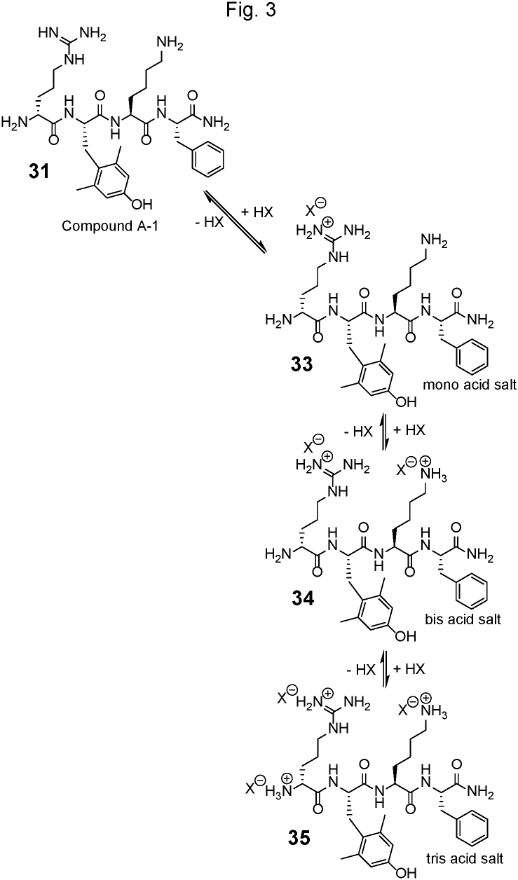

As used herein, the term “pharmaceutically acceptable salt” refers to a salt of a therapeutically active compound (e.g. a peptide or mixture of peptides) that can be prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts. These salts may be prepared by methods known to those skilled in the art. Other pharmaceutically acceptable carriers known to those of skill in the art are suitable for the present technology. In some embodiments, the compound is a zwitterion (an intramolecular salt). Exemplary salt forms of the peptide H-D-Arg-2′6′-Dmt-Lys-Phe-OH (A-2) are illustrated in FIG. 2. Exemplary salt forms of the peptide H-D-Arg-2′6′-Dmt-Lys-Phe-NH2 (A-1) are illustrated in FIG. 3.

As used herein, “phosphorodiamidate morpholino oligomers (PMOs)” refer to synthetic oligomers comprising a natural nucleobase linked to methylenemorpholine rings linked through phosphorodiamidate groups instead of a phosphate backbone. See: Summerton JE (2017). “Invention and Early History of Morpholinos: From Pipe Dream to Practical Products”. Morpholino Oligomers. Methods in Molecular Biology. 1565. Humana Press (Springer). pp. 1-15.

As used herein the term “prevent”, “prevents”, “preventing” or “prevention” refers to, in a statistical sample, reducing the occurrence of a disorder, symptom, condition or indication in a treated sample relative to an untreated control sample.

As used herein the term “prophylactic” refers to an action intended to prevent a disorder, symptom, condition or indication from occurring.

As used herein, the term “solvate” refers to forms of a compound (e.g. a peptide or mixture of peptides) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, the terms “subject” and “patient” are used interchangeably.

As used herein the term “synergistic therapeutic effect” refers to a greater-than-additive therapeutic effect which is produced by a combination of at least two therapeutic agents, and which exceeds that which would otherwise result from the individual administration of the agents. For example, lower doses of one or more therapeutic agents may be used in treating MD (e.g. DMD or BMD), resulting in increased therapeutic efficacy and decreased side-effects.

As used herein, the term “tautomer” refers to compounds (e.g. a peptide or mixture of peptides) that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, when referring to cardiac function parameters such as stroke volume, ejection fraction, shortening fraction and cardiac output, we refer to the left ventricle stroke volume, left ventricle ejection fraction, left ventricle fraction shortening and left ventricle cardiac output.

As used herein, the terms fractional shortening and shortening fraction are interchangeable.

### DETAILED DESCRIPTION

In one aspect, the present disclosure provides methods for treating cardiomyopathy or inhibiting the onset or progression of cardiomyopathy in a mammalian subject suffering from muscular dystrophy (such as Duchene muscular dystrophy or Becker muscular dystrophy), comprising administering to the subject in need thereof a therapeutically effective amount of a peptide or mixture of peptides as described in more detail below, or a pharmaceutically acceptable salt, hydrate, solvate and/or tautomer thereof. Generally, the mammalian subject will harbor a genetic permutation that affects the production and/or function of dystrophin protein. In some embodiments of the method, the genetic permutation is an insert, deletion, duplication, frameshift, or nonsense mutation related to the production of dystrophin protein. Said peptide or mixture of peptides can be administered alone, in a composition or formulation (e.g. medicament) and/or in combination with one or more additional therapeutic agents/drugs (i.e. active ingredients). In some embodiments, the subject is human.

The peptide or peptides within a mixture of peptides can be of generic Formula A:

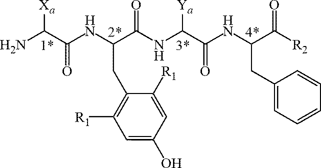

or a pharmaceutically acceptable salt, hydrate, solvate and/or tautomer thereof, wherein, each R₁ is independently H or —CH₃; R₂ is —OH or —NH₂; Xₐ and Yₐ are each independently selected from

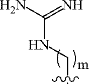

and

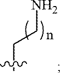

each m is 2, 3 or 4; each n is independently 1, 2, or 3; and the absolute stereochemistry at each of stereocenters 1^{∗}, 2^{∗}, 3^{∗}, and 4^{∗} is independently D or L. For example, the peptide can be of formula A-1:

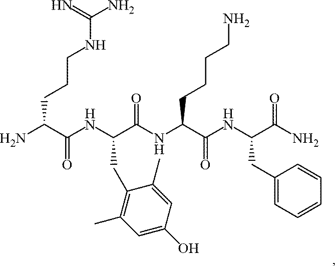

or a pharmaceutically acceptable salt, hydrate, solvate and/or tautomer thereof. The peptide can be of formula A-2:

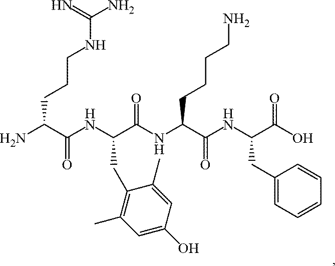

or a pharmaceutically acceptable salt, hydrate, solvate and/or tautomer thereof. The peptide can be of formula A-3:

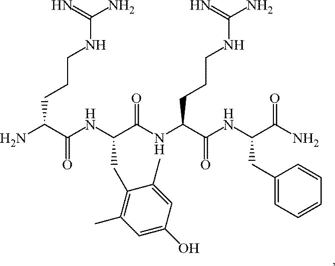

or a pharmaceutically acceptable salt, hydrate, solvate and/or tautomer thereof. The peptide can be of formula A-4:

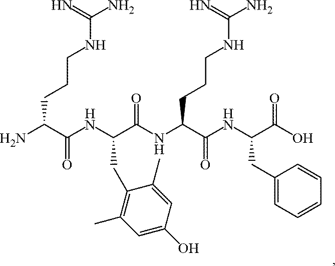

or a pharmaceutically acceptable salt, hydrate, solvate and/or tautomer thereof. The peptide can be of formula A-5:

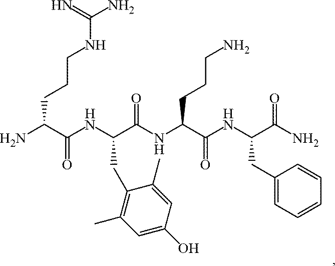

or a pharmaceutically acceptable salt, hydrate, solvate and/or tautomer thereof. The peptide can be of formula A-6:

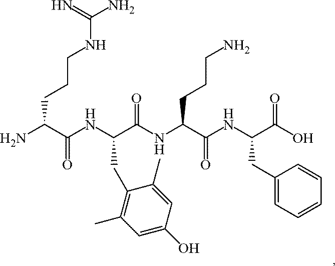

or a pharmaceutically acceptable salt, hydrate, solvate and/or tautomer thereof. The peptide can be of formula A-7:

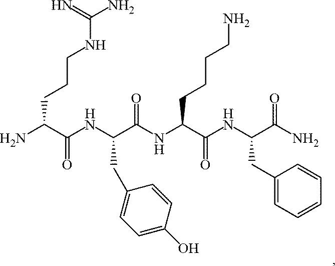

or a pharmaceutically acceptable salt, hydrate, solvate and/or tautomer thereof. The peptide can be of formula A-8:

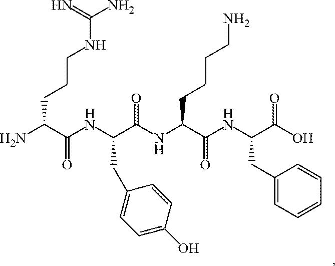

or a pharmaceutically acceptable salt, hydrate, solvate and/or tautomer thereof. The peptide can be administered individually or as a mixture comprising two or more of the peptides as defined herein. As noted, the peptide or mixture of peptides can be administered alone, in a formulation (e.g. medicament) or in combination with one or more other active ingredients. In some embodiments, the pharmaceutically acceptable salt can be selected from a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt.

As noted in the introduction, heart disease and cardiomyopathies are an expected, progressively debilitating condition associated with muscular dystrophy. Thus, in some embodiments, this disclosure relates to methods for treating, inhibiting, preventing, reducing, ameliorating or delaying the onset of signs, symptoms, or severity of cardiomyopathies associated with muscular dystrophy in a subject. In some embodiments, the method comprises administration of a peptide or mixture of peptides to a subject prophylactically prior to the detectable onset of a cardiomyopathy to thereby delay the detectable onset of or the progression of the cardiomyopathy. In some embodiments, the peptide or mixture of peptides can be administered to slow the progression of the cardiomyopathy or lessen the severity of the cardiomyopathy. In some embodiments, the peptide or mixture of peptides can be administered to reverse the physiological impact of the cardiomyopathy (e.g. reduce the size or thickness of the left ventricle wall). In some embodiments, the peptide or mixture of peptides can be administered to reverse the impact, occurrence or severity of cardiac arrythmias. In some embodiments, the peptide or mixture of peptides can be administered to inhibit fibrosis of the heart, delay the onset of fibrosis of the heart or reduce the extent of fibrosis of the heart of the subject. In some embodiments, the cardiomyopathy is hypertrophic cardiomyopathy. In some embodiments, the cardiomyopathy is dilated cardiomyopathy. In some embodiments, the condition to be addressed is progressive heart failure.

Administration of the peptide (or mixture of peptides) may exhibit various beneficial effects on the heart of the subject to which the peptide (or mixture of peptides) is administered. For example, administration of the peptide may increase the ejection fraction of the subject (or group of subjects) relative to an untreated subject (or untreated control group of subjects). Administration of the peptide (or mixture of peptides) may increase the shortening fraction (also sometimes referred to in the art as fractional shortening) of the subject (or group of subjects) relative to an untreated subject (or untreated control group of subjects). Administration of the peptide (or mixture of peptides) may increase the stroke volume of the subject (or group of subjects) relative to an untreated subject (or untreated control group of subjects). Administration of the peptide (or mixture of peptides) may increase the cardiac output of the subject (or group of subjects) relative to an untreated subject (or untreated control group of subjects). Administration of the peptide (or mixture of peptides) may increase two or more of: (i) ejection fraction; (ii) shortening fraction; (iii) stroke volume and (iv) cardiac output of the subject (or group of subjects) relative to an untreated subject (or untreated control group of subjects). Administration of the peptide (or mixture of peptides) may delay the onset of any decrease in one or more of: (i) ejection fraction; (ii) shortening fraction; (iii) stroke volume and (iv) cardiac output of the subject (or group of subjects) diagnosed with muscular dystrophy relative to an untreated subject (or untreated control group of subjects). Administration of the peptide (or mixture of peptides) may delay the onset of, or delay the progression of, cardiac fibrosis of the subject (or group of subjects) relative to an untreated subject (or untreated control group of subjects).

The peptide, mixture of peptides, and/or other therapeutic agent(s)/drug(s) can be administered by any known or future developed mode of administration. For example, administration can be oral. Administration can be systemic. Administration can be subcutaneous. Administration can be intravenous. Administration can be topical, intraperitoneal, intradermal, transdermal, ophthalmical, intrathecal, intracerebroventricular, iontophoretical, transmucosal, intravitreal, intranasal, or intramuscular. In some embodiments, peptide, mixture of peptides and/or the other therapeutic agent(s)/drug(s) are separately, sequentially or simultaneously administered. In some embodiments, administration of the peptide or mixture of peptides with another therapeutic agent produces a synergistic therapeutic effect.

In some embodiments, the peptide or mixture of peptides is administered to the subject for 6 weeks or more. In some embodiments, the peptide or mixture of peptides is administered to the subject for 12 weeks or more. In some embodiments, the peptide or mixture of peptides is administered to the subject for 24 weeks or more. In some embodiments, the peptide or mixture of peptides is administered to the subject for 48 weeks or more. In some embodiments, the peptide or mixture of peptides is administered to the subject for 72 weeks or more. In some embodiments, the peptide or mixture of peptides is administered to the subject for 96 weeks or more. In some embodiments, the peptide or mixture of peptides is administered to the subject for 2 years or more. In some embodiments, the peptide or mixture of peptides is administered to the subject for 3 years or more. In some embodiments, the peptide or mixture of peptides is administered until no continued therapeutic benefit is observed. In some embodiments, the peptide or mixture of peptides is administered until the end of life or near end of life of the subject. In some embodiments, the subject is a human and administration of the peptide begins as soon as symptoms of muscular dystrophy are diagnosed or observed and continues throughout the lifetime of the subject.

The peptide or mixture of peptides can be administered at any reasonable interval. The interval of administration (i.e. dosing) will depend on several factors including the mode of administration, the dose to be administered, the formulation of the active ingredients, the toxicity of the formulation and any allergies or other traits of the subject. Those of skill in the art will be able to determine the proper interval for dosing. In some embodiments, dosing will occur about once per day. In some embodiments, dosing will occur about twice per day. In some embodiments, dosing will occur about thrice per day. In some embodiments, dosing will occur about once every other day. In some embodiments, dosing will occur about once per week. In some embodiments, dosing will occur about once every other week. In some embodiments, dosing will occur about once per month. In some embodiments, dosing will occur about once every other month. In some embodiments, dosing will occur about once every three months. In some embodiments, dosing will occur about once every six months. In some embodiments, dosing will occur about once every nine months. In some embodiments, dosing will occur about once every year. In some embodiments, the peptide or mixture of peptides is administered as a depot formulation comprising a peptide or peptides that are encapsulated by, or disposed within, silica microparticles.

As noted in the introduction, there are many established treatments/medications used to address the symptoms of cardiomyopathy associated with MD. These include, for example, administration of corticosteroids (e.g. bethamethasone, prednisone, prednisolone, triamcinolone, methylprednisolone or dexamethasone), administration of angiotensinconverting enzyme (ACE) inhibitors (e.g. benazepril, daptopril, enalapril, fosinopril, lisinopril, moexipril, perindopril, quinapril, ramipril or trandolapril), administration of beta blockers (e.g. acebutolol, atenolol, bisoprolol, metoprolol, nadolol, nebivolol or propranolol), administration of angiotensin receptor blockers (ARBs, e.g. azilsartan, candesartan, eprosartan, irbesartan, losartan, olmesartan, telmisartan and valsartan) and/or administration of a medication that increases the production of dystrophin in muscle (for example, those subjects being treated with a phosphorodiamidate morpholino oligomer (PMO) such as Eteplirsen (Exondys 51®), Golodirsen (Vyondys 53™) or Casimersen (Amondys 45™) or a PPMO). Thus, in some embodiments, the methods described herein can be further directed to administration of the peptide or mixture of peptides (as defined in more detail below) in combination with one or more of the following therapeutic agents: (i) a corticosteroid; (ii) an ACE inhibitor; (iii) an ARB, (iv) a beta blocker; and (v) a medication that increases the production of dystrophin in muscle (e.g. a phosphorodiamidate morpholino oligomer (PMO) such as Exondys 51® (Eteplirsen), Golodirsen (Vyondys 53™)) or Casimersen (Amondys 45™) or PPMO).

Therefore, in some embodiments, the methods described herein can be further directed to administration of the peptide or mixture of peptides described herein in combination with a medication that increases the production of dystrophin in muscle. For example, in some embodiments, the methods described herein can be further directed to administration of the peptide or mixture of peptides in combination with Exondys 51® (Eteplirsen). Further, in some embodiments, the methods described herein can be further directed to administration of the peptide or mixture of peptides in combination with Vyondys 53™ (Golodirsen). Further, in some embodiments, the methods described herein can be further directed to administration of the peptide or mixture of peptides in combination with Amondys 45™ (Casimersen). In some embodiments, the peptide administered in combination with a medication that increases the production of dystrophin in muscle (e.g. a phosphorodiamidate morpholino oligomer (PMO) such as Exondys 51® (Eteplirsen), Golodirsen (Vyondys 53™) or Amondys 45™ (Casimersen) or PPMO) is H-D-Arg-2′6′-Dmt-Lys-Phe-NH₂ or its carboxylate form, H-D-Arg-2′6′-Dmt-Lys-Phe-OH (or a pharmaceutically acceptable salt, hydrate, solvate and/or tautomer of either of the foregoing). Such combination of drugs may be synergistic because whilst the medication that increases production of dystrophin in muscle improves the subject’s ability to walk and exercise, administration of the peptide (or mixture of peptides) may relieve at least some of the added stress applied to the heart by this increased physical activity and thereby inhibit or delay onset of and/or progression of heart disease and/or cardiomyopathies associated with muscular dystrophy. The combination of drugs might also be considered synergistic where it allows the subject to receive higher doses of the medication that increases production of dystrophin without accompanying deleterious effects on the heart of the subject. The combination of drugs might also be considered synergistic where it improves cardiac function as well as improves skeletal muscle function and ambulation in the subject beyond what is observed by administration of each drug individually. For example, where the peptide or mixture of peptides addresses mitochondrial dysfunction and thereby increase ATP available to power the muscles (including heart muscle) whilst other drugs (e.g. Exondys 51® (Eteplirsen), Golodirsen (Vyondys 53™) or Amondys 45™ (Casimersen)) increase dystrophin levels in the muscles thereby improving overall muscle structure and function. Methods & Treatments:

In one aspect, the present disclosure provides methods for treating the signs, symptoms, or severity of cardiomyopathy in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) and ARB, (iv) a beta blocker; and (v) a medication that increases the production of dystrophin in muscle (e.g. a phosphorodiamidate morpholino oligomer (PMO) such as Exondys 51® (Eteplirsen), Golodirsen (Vyondys 53™)) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

In another aspect, the present disclosure provides methods for inhibiting the signs, symptoms, or severity of cardiomyopathy in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) a beta blocker; and (iv) a medication that increases the production of dystrophin in muscle (e.g. a phosphorodiamidate morpholino oligomer (PMO) such as Exondys 51® (Eteplirsen), Golodirsen (Vyondys 53™)) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

In still another aspect, the present disclosure provides methods for preventing the signs, symptoms, or severity of cardiomyopathy in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) and ARB, (iv) a beta blocker; and (v) a medication that increases the production of dystrophin in muscle (e.g. a phosphorodiamidate morpholino oligomer (PMO) such as Exondys 51® (Eteplirsen), Golodirsen (Vyondys 53™)) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

In yet another aspect, the present disclosure provides methods for ameliorating the signs, symptoms, or severity of cardiomyopathy in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) an ARB; (iv) a beta blocker; and (v) a medication that increases the production of dystrophin in muscle (e.g. a phosphorodiamidate morpholino oligomer (PMO) such as Exondys 51® (Eteplirsen), Golodirsen (Vyondys 53™)) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

In still a further aspect, the present disclosure provides methods for delaying the onset of the signs, symptoms, or severity of cardiomyopathy in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail below, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) a beta blocker; (iv) an ARB; and (v) a medication that increases the production of dystrophin in muscle (e.g. a phosphorodiamidate morpholino oligomer (PMO) such as Exondys 51® (Eteplirsen), Golodirsen (Vyondys 53™) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

In yet another aspect, the present disclosure provides methods for delaying the onset of cardiac fibrosis in a mammalian subject suffering from muscular dystrophy (such as Duchene muscular dystrophy or Becker muscular dystrophy), comprising administering to the subject in need thereof a therapeutically effective amount of a therapeutically active peptide or mixture of peptides as described in more detail below, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides (as defined herein) in combination with one or more of the following therapeutic agents: (i) a corticosteroid; (ii) an ACE inhibitor; (iii) a beta blocker; (iv) an ARB; and (v) a medication that increases the production of dystrophin in muscle (e.g. a phosphorodiamidate morpholino oligomer (PMO) such as Exondys 51® (Eteplirsen), Golodirsen (Vyondys 53™) or Casimersen (Amondys 45™) or a PPMO). In some embodiments, co-administration is simultaneous, such as by simultaneous administration by IV injection. In some embodiments, co-administration is simultaneous, but by different routes of administration, such as by administering a medication that increases the production of dystrophin in muscle by IV injection while the peptide or mixture of peptides is/are administered by, for example, subcutaneous injection or other route of administration of a long-term systemic release depot formulation.

A mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a non-human primate. In some embodiments, the mammal is a human.

Said peptide or mixture of peptides can be administered alone, in a composition or formulation or in combination with one or more additional therapeutic agents. In some embodiments, the compositions are used as medicaments or in the preparation of medicaments for: (i) treating the signs, symptoms, or severity of cardiomyopathy in a mammalian subject suffering from muscular dystrophy (such as Duchene muscular dystrophy or Becker muscular dystrophy); (ii) inhibiting the signs, symptoms, or severity of cardiomyopathy in a mammalian subject suffering from muscular dystrophy (such as Duchene muscular dystrophy or Becker muscular dystrophy); (iii) preventing the signs, symptoms, or severity of cardiomyopathy in a mammalian subject suffering from muscular dystrophy (such as Duchene muscular dystrophy or Becker muscular dystrophy); (iv) ameliorating the signs, symptoms, or severity of cardiomyopathy in a mammalian subject suffering from muscular dystrophy (such as Duchene muscular dystrophy or Becker muscular dystrophy); or (v) delaying the onset the signs, symptoms, or severity of cardiomyopathy in a mammalian subject suffering from muscular dystrophy (such as Duchene muscular dystrophy or Becker muscular dystrophy). In some embodiments, the compositions are used as medicaments or in the preparation of medicaments for reducing the amount of and/or delaying the onset of cardiac fibrosis in a subject. In some embodiments, the use further comprises, using in combination with the aforementioned medicament(s) for treating, inhibiting, preventing, ameliorating or delaying the onset of the signs, symptoms, or severity of cardiomyopathy in a mammalian subject suffering from muscular dystrophy, a further medicament that increases the production of dystrophin in muscle (e.g. a medicament comprising a phosphorodiamidate morpholino oligomer (PMO) such as Exondys 51® (Eteplirsen), Golodirsen (Vyondys 53™) or Casimersen (Amondys 45™) or a PPMO). In some embodiments, the subject is a human.

In some embodiments, the peptide of mixture of peptides is/are administered in a depot formulation (discussed below), such as a silica-based depot formulation, wherein the peptide or peptides are encapsulated/encased in silica particles (nanoparticles or microparticles) that slowly release the peptide or peptides over time (e.g. by sustained and/or controlled release over days, weeks or months). For example, the depot formulation of the peptides may be injected subcutaneously to provide for long-term systemic release of the peptide or peptides to the subject.

Administration of the peptide (or mixture of peptides) or a composition comprising the peptide(s) may exhibit various beneficial effects on the heart of the subject to which the peptide (or mixture of peptides) or composition is administered. For example, administration of the peptide(s) or composition may increase the ejection fraction of the subject (or group of subjects) relative to an untreated subject (or untreated control group of subjects). Administration of the peptide(s) or composition may increase the shortening fraction of the subject (or group of subjects) relative to an untreated subject (or untreated control group of subjects). Administration of the peptide(s) or composition may increase the stroke volume of the subject (or group of subjects) relative to an untreated subject (or untreated control group of subjects). Administration of the peptide(s) or composition may increase the cardiac output of the subject (or group of subjects) relative to an untreated subject (or untreated control group of subjects). Administration of the peptide(s) or composition may decrease or delay the onset of cardiac fibrosis of the subject (or group of subjects) relative to an untreated subject (or untreated control group of subjects). Administration of the peptide(s) or composition may increase two or more of: (i) ejection fraction; (ii) shortening fraction; (iii) stroke volume; (iv) cardiac output; and (v) cardiac fibrosis of the subject (or group of subjects) relative to an untreated subject (or untreated control group of subjects). Administration of the peptide (or mixture of peptides) or composition may delay the onset of any decrease in one or more of: (i) ejection fraction; (ii) shortening fraction; (iii) stroke volume; and (iv) cardiac output; or delay the onset of cardiac fibrosis of the subject (or group of subjects) diagnosed with muscular dystrophy relative to an untreated subject (or untreated control group of subjects).

### Peptides & Mixtures of Peptides

The aforementioned methods can be practiced with peptides or mixtures of peptides. Peptides suitable for use in the aforementioned methods are peptides of generic Formula A or a pharmaceutically acceptable salt, hydrate, solvate and/or tautomer thereof, wherein Formula A is:

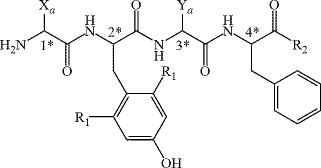

wherein, each R₁ is independently H or —CH₃; R₂ is —OH or —NH₂; Xₐ and Yₐ are each independently selected from

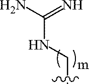

and

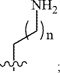

each m is 2, 3 or 4; each n is independently 1, 2, or 3; and the absolute stereochemistry at each of stereocenters 1*, 2*, 3*, and 4* is independently D or L.

In some embodiments, the peptide of generic Formula A is a peptide of formula A-1:

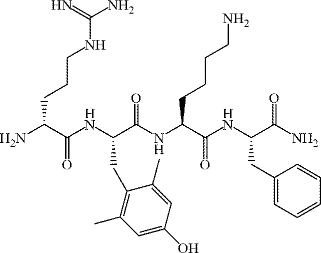 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-2:

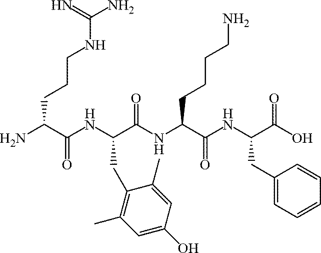 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-3:

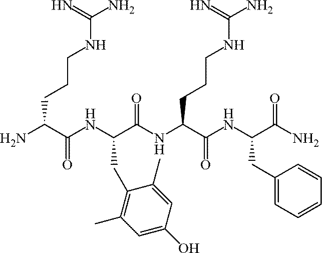 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-4:

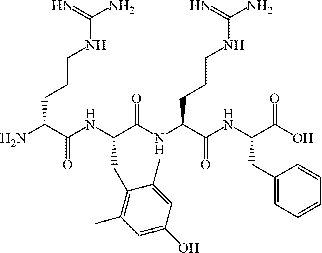 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-5:

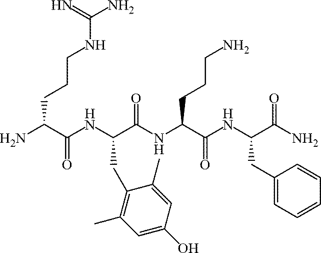 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-6:

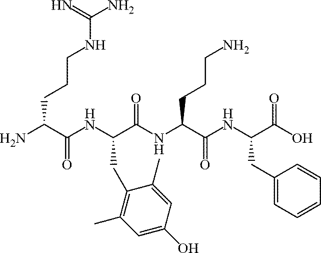 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O]

or a pharmaceutically acceptable salt, hydrate, solvate and/or tautomer thereof.

In some embodiments, the peptide of generic Formula A is a peptide of formula A-7:

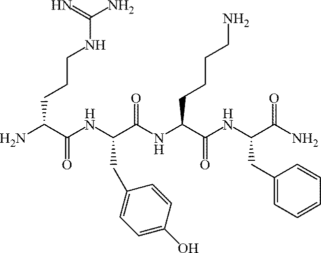 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O]

or a pharmaceutically acceptable salt, hydrate, solvate and/or tautomer thereof.

In some embodiments, the peptide of generic Formula A is a peptide of formula A-8:

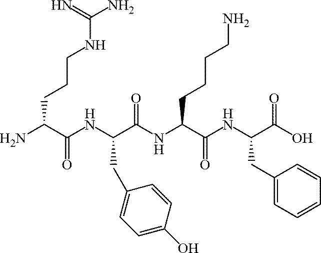 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]

or a pharmaceutically acceptable salt, hydrate, solvate and/or tautomer thereof.

In some embodiments, mixtures of two or more of the above described peptides are used as a/the therapeutic agent. Such mixtures may be present intentionally (e.g. by mixing the peptides post synthesis) or fortuitously (e.g. by the hydrolysis of a C-terminal amide to a C-terminal carboxylic acid). Whenever reference is made herein to a ‘peptide or mixture of peptides’, it is implied and intended that each individual peptide of said “peptide or mixture of peptides” can exist as a free acid/base, in zwitterionic form or in any salt form, including in a pharmaceutically acceptable salt form (e.g. See: FIGS. 2 & 3 for various salt forms of A-1 & A-2).

### Peptide Synthesis

The peptides may be synthesized by any of the methods well known in the art. The peptides can be prepared using solid-phase synthesis methodology. The peptides can be synthesized by using solution-phase methodology. Suitable methods for chemically synthesizing the peptides include, for example, those described in any of WO 2004/070054, WO 2018/03490, WO 2019/099481, WO 2018/187400 or WO 2019/118878. In some embodiments, the peptides are C-terminal amides and in some embodiments the peptide are C-terminal carboxylic acids. Peptides that are C-terminal amides can be converted to peptides comprising C-terminal acids by simple hydrolysis as described in Example 5, below.

For example, the peptides disclosed herein can be prepared using any peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp.1 to 19; Stewart et al., Solid-Phase Peptide Synthesis (1989) W. H.; Houghten, Proc. Natl. Acad. Sci. USA (1985) 82: p.5132; Stuart and Young in Solid Phase Peptide Synthesis, Second Edition, Pierce Chemical Company (1984), and in Methods Enzymol., 289, Academic Press, Inc., New York (1997)). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. Typically in solid phase synthesis, after coupling, a capping step is performed to cap (render unreactive) any unreacted amine groups. These steps (i.e., deprotection, coupling, and optionally capping) may be repeated to form the desired peptide chain. Once the desired peptide chain is complete, the peptide may be cleaved from the solid support.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z, cbz or Cbz), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p- biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase or solution-phase peptide synthesis discussed herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), and its HCl salt (EDC•HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include (1-[Bis(dimethylamino)methylene]-1H-1,2,3-triazolo[4,5-b]pyridinium 3-oxide hexafluorophosphate (HATU) and (2-(1H-benzotriazol-1-yl)-1,1,3,3-tetramethyluronium hexafluorophosphate (HBTU), generally used in combination with an organic base such as N,N-diisopropylethylamine (DIEA) and a hindered pyridine-type base such as lutidine or collidine.

In some embodiments, the amino acids can be activated toward coupling by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996); and WO 2018/034901. Such methods of peptide synthesis may be used to produce the peptides disclosed herein either by solution-phase or solid-phase methodology.

### Salt Forms (Some of Which Are Pharmaceutically Acceptable Salts) & Other Forms

Compounds of Formula A (including without limitation A-1, A-2, A-3, A-4, A-5, A-6, A-7 and A-8) can exist in various forms, such as in salt form(s) (such as a pharmaceutically acceptable salt form), in tautomeric form(s), in solvated form(s) and/or in hydrate form(s).

For example, FIG. 2 illustrates various forms that Compound A-2 can take and FIG. 3 illustrates various forms that Compound A-1 can take. With reference to FIG. 2, (20) illustrates a mono-basic salt form of Compound A-2, wherein the C-terminal carboxylate has been ionized as its base-salt. As illustrated, the basic generic salt represented by YOH can ionize to produce Y+ and OH— and thereby ionize Compound A-2 (21) to form (20). The generic basic salt represented by YOH could be, for example, sodium hydroxide (NaOH), potassium hydroxide (KOH) or lithium hydroxide (LiOH). The mono-basic salt form (20) can be protonated with acid to form Compound A-2 (21). However, Compound A-2 (21) can also be represented in zwitterionic form (22) resulting from the internal distribution of a proton between the carboxylate and one of the basic groups. Compound A-2 ((21) or (22)) can be further protonated with a single equivalent of acid (e.g. represented by HX wherein H+ is the proton and X- represents the counterion and is embodied by acids such as HCl, HBr or HI) to thereby produce a mono-acid salt (23). The mono-acid salt (23) can be further acidified with another equivalent of acid to thereby produce a bis-acid salt (24). The bis-acid salt (24) can be further acidified with another equivalent of acid to thereby produce a tris-acid salt (25). One of skill in the art will appreciate that these transitions between the various salt forms are easily accomplished by use of an appropriate amount of acid or base. One of skill in the art will further appreciate that such transitions between salt forms are also applicable to any compounds represented by Formula A, including without limitation Compounds A-1, A-3, A-4, A-5, A-6, A-7 or A-8.

The peptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the pharmaceutically acceptable salt is a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt.

Certain compound(s)/peptide(s) disclosed in the present disclosure can exist in unsolvated forms as well as solvated forms, including hydrated forms. Solvated forms can exist, for example, because it is difficult or impossible to remove all the solvent from the peptide post synthesis. In general, the solvated forms are equivalent to unsolvated forms and are encompassed within the scope of the present disclosure.

Certain compound(s)/peptide(s) of the present disclosure may exist in crystalline form, multiple crystalline forms, amorphous forms or any combination of the foregoing. Certain compound(s)/peptide(s) of the present disclosure may exist in various tautomeric forms. Certain compound(s)/peptide(s) of the present disclosure may exist in various salt forms or mixtures of salt forms. In general, all physical forms of the compound(s)/peptide(s) disclosed herein are equivalent for the uses contemplated by the present disclosure and are intended to be within the scope of the present disclosure.

### Chiral/Stereochemistry Considerations

Peptides/compounds described herein can comprise one or more asymmetric centers, and thus can exist in various isomeric forms, e.g., enantiomers and/or diastereomers (i.e., stereoisomers). Chiral centers in illustrated structures (including the claims) may be identified herein by use of an asterisk (*). For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The disclosure of the present application additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess); as purity is a relative term in the sense that it is exceedingly difficult to achieve 100% purity. In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that for a “D″-amino acid the configuration is “R” and for an “L″-amino acid, the configuration is “S”. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91 % by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g. as compared with the other enantiomer). In certain embodiments, the weights are based upon total weight of all enantiomers or stereoisomers of the compound.

In the compositions provided herein, an enantiomerically pure compound (e.g. a peptide) can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the enantiomers of the compound. In certain embodiments, the active ingredient can be formulated with little or no excipient or carrier.

### Compositions, Formulations & Dosing

This disclosure further relates to compositions that can be used in the disclosed methods wherein the composition comprises at least one peptide of Formula A (e.g. Compound A-1, A-2, A-3, A-4, A-5, A-6, A-7 or A-8), but may also include or more of the following compounds/therapeutic agents: (i) a corticosteroid; (ii) an ACE inhibitor; (iii) a beta blocker; (iv) an ARB; and (v) a medication that increases the production of dystrophin in muscle (e.g. a phosphorodiamidate morpholino oligomer (PMO) such as Exondys 51® (Eteplirsen), Golodirsen (Vyondys 53™) or Casimersen (Amondys 45™) or a PPMO). Such a composition can be formed, for example, by dissolving or suspending the selected compound(s)/peptide (or mixture of peptides) in water, buffer, detergent, excipient, organic solvent or a mixture of two or more of the foregoing. In some embodiments, the composition can be prepared by dissolving or suspending the selected compound(s)/peptide(s) in water. In some embodiments, the composition can be prepared by dissolving or suspending the selected compound(s)/peptide(s) in buffer. In some embodiments, the composition can be prepared by dissolving or suspending the selected compound(s)/peptide(s) in excipient. In some embodiments, the composition can be prepared by dissolving or suspending the selected compound(s)/peptide(s) in a pharmaceutically acceptable carrier. In some embodiments, the composition or formulation is a medicament.

The peptide or mixture of peptides and optionally other therapeutic agents/drugs may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, phosphoric, monohydrogenphosphoric, dihydrogenphosphoric, sulfuric, monohydrogensulfuric, hydriodic, carbonic, monohydrogencarbonic, or phosphorous acids and the like, as well as the salts derived from organic acids like acetic, propionic, isobutyric, maleic, malonic, benzoic, succinic, suberic, fumaric, lactic, mandelic, oxalic, phthalic, benzenesulfonic, p-toluenesulfonic, citric, tartaric, methanesulfonic, trifluoroacetic, and the like. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present disclosure may contain both basic and acidic functionalities that allow the compounds to be converted into either base or acid addition salts (See for example: FIGS. 2, 3). These salts may be prepared by methods known to those skilled in the art. Other pharmaceutically acceptable carriers known to those of skill in the art are suitable for use with the present technology. Suitable buffering agents may include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In some embodiments, the compositions or formulations can be used as medicaments or in the preparation of medicaments for: (i) treating the signs, symptoms, or severity of cardiomyopathy in a mammalian subject suffering from muscular dystrophy (such as Duchene muscular dystrophy or Becker muscular dystrophy); (ii) inhibiting the signs, symptoms, or severity of cardiomyopathy in a mammalian subject suffering from muscular dystrophy (such as Duchene muscular dystrophy or Becker muscular dystrophy); (iii) preventing the signs, symptoms, or severity of cardiomyopathy in a mammalian subject suffering from muscular dystrophy (such as Duchene muscular dystrophy or Becker muscular dystrophy); (iv) ameliorating the signs, symptoms, or severity of cardiomyopathy in a mammalian subject suffering from muscular dystrophy (such as Duchene muscular dystrophy or Becker muscular dystrophy); or (v) delaying the onset of the signs, symptoms, or severity of cardiomyopathy in a mammalian subject suffering from muscular dystrophy (such as Duchene muscular dystrophy or Becker muscular dystrophy). In some embodiments, the compositions or formulations can be used as medicaments or in the preparation of medicaments for reducing the amount of or delaying the onset of cardiac fibrosis. In some embodiments, the use further comprises, using in combination with the aforementioned medicament(s) for treating, inhibiting, preventing, ameliorating or delaying the onset of the signs, symptoms, or severity of cardiomyopathy in a mammalian subject suffering from muscular dystrophy, a further medicament that increases the production of dystrophin in muscle (e.g. a medicament comprising a phosphorodiamidate morpholino oligomer (PMO) such as Exondys 51® (Eteplirsen), Golodirsen (Vyondys 53™)) or Casimersen (Amondys 45™) or a PPMO).

The compositions and methods of the present disclosure may be utilized to treat an individual/subject in need thereof. In certain embodiments, the individual is a mammal such as a human, or a non-human mammal. When administered to an animal, such as a human, the composition or the compound/peptide is preferably administered as a pharmaceutical composition comprising, for example, a peptide or mixture of peptides and an excipient or pharmaceutically acceptable carrier.

As stated above, an “effective amount” refers to any amount of the active compound (e.g. a peptide or mixture of peptides; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to treat the particular condition or disease of a particular subject. The effective amount for any particular indication can vary depending on such factors as the disease or condition being treated, the particular compound of the present application being administered, the size of the subject, or the severity of the disease or condition. The effective amount may be determined during pre-clinical trials and/or clinical trials by methods familiar to physicians and clinicians. One of ordinary skill in the art can empirically determine the effective amount of a particular peptide or mixture of peptides of the present application and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient’s peak or sustained plasma level of the drug. “Dose” and “dosage” are used interchangeably herein. A dose may be administered by oneself, by another or by way of a device (e.g. a pump).

For any compound (e.g. a peptide or mixture of peptides) described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Peptides/compounds (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, any of the animal model system known in the art can be used prior to administration to human subjects.

Dosage, toxicity and therapeutic efficacy of any therapeutic peptides, compounds, compositions (e.g. formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography, optionally coupled with mass spectroscopy detection (e.g. LC/MS).

The effective amount may be determined during pre-clinical trials and clinical trials by methods familiar to physicians and clinicians. An effective amount of the compound(s)/peptide(s) useful in the methods disclosed herein may be administered to a mammal in need thereof by any of a number of well-known methods for administering pharmaceutical compounds. The peptide may be administered systemically or locally.

Typically, an effective amount of the compound(s)/peptide(s), sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of a peptide can range from 0.001-10,000 micrograms per kg body weight. In one embodiment, peptide concentrations in a carrier range from 0.2 to 2000 micrograms per delivered milliliter. An exemplary treatment regime entails administration once per day or once a week.

In some embodiments, a therapeutically effective amount of peptide may be defined as a concentration of peptide at the target tissue (e.g. heart tissue) of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

In certain embodiments, intravenous administration of a compound (e.g. a peptide or mixture of peptides) may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

In certain embodiments, subcutaneous administration of a compound (e.g. a peptide or mixture of peptides) may typically be from 0.1 mg/kg/day to 20 mg/kg/day. In one embodiment, subcutaneous administration of a compound may typically be from 0.1 mg/kg/day to 2 mg/kg/day. In one embodiment, subcutaneous administration of a compound may typically be from 0.5 mg/kg/day to 5 mg/kg/day. In one embodiment, subcutaneous administration of a compound may typically be from 1 mg/kg/day to 20 mg/kg/day. In one embodiment, subcutaneous administration of a compound may typically be from 1 mg/kg/day to 10 mg/kg/day. In one embodiment, subcutaneous administration of a compound may typically be from 0.5 mg/kg/day to 1.0 mg/kg/day. In some embodiments, subcutaneous administration of a compound may typically be 10 mg/kg/day. In some embodiments, subcutaneous administration of a compound may typically be 9 mg/kg/day. In some embodiments, subcutaneous administration of a compound may typically be 8 mg/kg/day. In some embodiments, subcutaneous administration of a compound may typically be 7 mg/kg/day. In some embodiments, subcutaneous administration of a compound may typically be 6 mg/kg/day. In some embodiments, subcutaneous administration of a compound may typically be 5 mg/kg/day. In some embodiments, subcutaneous administration of a compound may typically be 4 mg/kg/day. In some embodiments, subcutaneous administration of a compound may typically be 3 mg/kg/day. In some embodiments, subcutaneous administration of a compound may typically be 2 mg/kg/day. In some embodiments, subcutaneous administration of a compound may typically be 1 mg/kg/day. In some embodiments, subcutaneous administration of a compound may typically be 0.9 mg/kg/day. In some embodiments, subcutaneous administration of a compound may typically be 0.8 mg/kg/day. In some embodiments, subcutaneous administration of a compound may typically be 0.75 mg/kg/day. In some embodiments, subcutaneous administration of a compound may typically be 0.7 mg/kg/day. In some embodiments, subcutaneous administration of a compound may typically be 0.6 mg/kg/day. In some embodiments, subcutaneous administration of a compound may typically be 0.5 mg/kg/day. In some embodiments, subcutaneous administration of a compound may typically be 0.4 mg/kg/day. In some embodiments, subcutaneous administration of a compound may typically be 0.3 mg/kg/day. In some embodiments, subcutaneous administration of a compound may typically be 0.25 mg/kg/day. In some embodiments, subcutaneous administration of a compound may typically be 0.2 mg/kg/day. In some embodiments, subcutaneous administration of a compound may typically be 0.1 mg/kg/day. Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous dose per day administration would be from one order to several orders of magnitude lower. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

The skilled artisan will appreciate that certain factors may influence the dosage, mode of administration and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The peptide, mixture of peptides or other therapeutic agent(s)/drug(s) can be administered by any known or future developed mode of administration. For example, administration can be oral. Administration can be systemic. Administration can be subcutaneous. Administration can be intravenous. Administration can be topical, intraperitoneal, intradermal, transdermal, ophthalmical, intrathecal, intracerebroventricular, iontophoretical, transmucosal, intravitreal, intranasal, or intramuscular. In some embodiments, peptide or mixture of peptides and the other therapeutic agent(s)/drug(s) are separately, sequentially or simultaneously administered. In some embodiments, administration of the peptide or mixture of peptides with another therapeutic agent produces a synergistic therapeutic effect.

In some embodiments, the peptide or mixture of peptides is administered to the subject for 6 weeks or more. In some embodiments, the peptide or mixture of peptides is administered to the subject for 12 weeks or more. In some embodiments, the peptide or mixture of peptides is administered to the subject for 24 weeks or more. In some embodiments, the peptide or mixture of peptides is administered to the subject for 48 weeks or more. In some embodiments, the peptide or mixture of peptides is administered to the subject for 72 weeks or more. In some embodiments, the peptide or mixture of peptides is administered to the subject for 96 weeks or more. In some embodiments, the peptide or mixture of peptides is administered to the subject for 2 years or more. In some embodiments, the peptide or mixture of peptides is administered to the subject for 3 years or more. In some embodiments, the peptide or mixture of peptides is administered until no continued therapeutic benefit is observed. In some embodiments, the peptide or mixture of peptides is administered until the end of life of the subject.

The peptide or mixture of peptides can be administered at any reasonable interval. The interval of administration (i.e. dosing) will depend on several factors including the mode of administration, the dose to be administered, the formulation of the active ingredients, the toxicity of the formulation and any allergies or other traits of the subject. Those of skill in the art will be able to determine the proper interval for dosing. In some embodiments, dosing will occur about once per day. In some embodiments, dosing will occur about twice per day. In some embodiments, dosing will occur about thrice per day. In some embodiments, dosing will occur about once every other day. In some embodiments, dosing will occur about once per week. In some embodiments, dosing will occur about once every other week. In some embodiments, dosing will occur about once per month. In some embodiments, dosing will occur about once every other month. In some embodiments, dosing will occur about once every three months. In some embodiments, dosing will occur about once every six months. In some embodiments, dosing will occur about once every nine months. In some embodiments, dosing will occur about once every year.

The pharmaceutical compositions (e.g. a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g. a formulation or medicament) used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 2, 3, 4, 5, 6, 7 days, weeks, months or more of treatment).

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound (e.g. a peptide or mixture of peptides) of the present application can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g. a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and must be preserved against the contaminating action of microorganisms such as bacteria and fungi.

Sterile injectable solutions (e.g. a formulation or medicament) can be prepared by incorporating the active compound (e.g. a peptide or mixture of peptides) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

The therapeutic compounds (e.g. a peptide or mixture of peptides) or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include aqueous solutions of the active compounds (e.g. a peptide or mixture of peptides) in water-soluble form. Additionally, suspensions of the therapeutic compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the therapeutic compounds to allow for the preparation of highly concentrated solutions.

For oral administration, the compounds (e.g. a peptide or mixture of peptides) can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above may be chemically modified so that oral delivery of the derivative is efficacious. Generally, the chemical modification contemplated is the attachment of at least one moiety to the therapeutic agent(s), ingredient(s), and/or excipient(s), where said moiety permits: (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound (e.g. a peptide or mixture of peptides) or pharmaceutical composition can be included in the formulation as fine multi-particulates in the form of granules or pellets of particle size about 1-2 mm. The formulation of the material for capsule administration could also be as a powder, lightly compressed plugs or even as tablets. The therapeutic compound or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound or pharmaceutical composition with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of the therapeutic compound (e.g. a peptide or mixture of peptides) or composition into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The compounds, peptides, peptide mixtures and compositions disclosed herein can be included in a formulation as fine multi-particulates in the form of granules or pellets of particle size about 1 mm. The formulation of the material for capsule administration could also be as a powder, lightly compressed plugs or even as tablets. The formulation could be prepared by compression.

For buccal administration, the compositions may take the form of tablets or lozenges formulated in conventional manner.

For topical administration, a compound, peptide or mixture of peptides may be formulated as solutions, gels, ointments, creams, suspensions, etc. as are well-known in the art.

For administration by inhalation, peptides, compounds or compositions (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound and a suitable powder base such as lactose or starch.

Nasal delivery of a therapeutic compound (e.g. a peptide or mixture of peptides) or pharmaceutical composition of the present application is also contemplated. Nasal delivery allows the passage of a therapeutic compound or pharmaceutical composition of the present application to the blood stream directly after administering the therapeutic compound or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound or pharmaceutical composition.

Alternatively, the therapeutic compound (e.g. a peptide or mixture of peptides) or pharmaceutical composition may be in powder form for constitution with a suitable vehicle, e.g., sterile pyrogen-free water, before use.

Also contemplated herein is pulmonary delivery of the compounds, peptide or mixture of peptides disclosed herein (or salts, hydrates, solvates and/or tautomers thereof). The compound, peptide or mixture of peptides is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the compound, peptides or mixtures of peptides disclosed herein. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro. Chemically modified compound may also be prepared in different formulations depending on the type of chemical modification or the type of device employed.

Formulations suitable for use with a nebulizer, either jet or ultrasonic, will typically comprise a peptide or mixture of peptides disclosed herein dissolved in water at a concentration of about 0.1 to 25 mg of biologically active compound (e.g. a peptide or mixture of peptides) per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the peptide or mixture of peptides disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a peptide or mixture of peptides disclosed herein and may also include a bulking agent, such as lactose, sorbitol, sucrose, trehalose, or mannitol in amounts which facilitate dispersal of the powder from the device, e.g., 50 to 90% by weight of the formulation. The compound (or derivative) should advantageously be prepared in particulate form with an average particle size of less than 10 micrometers (µm), most preferably 0.5 to 5 µm, for most effective delivery to the deep lung.

In addition to the formulations described above, a peptide or mixture of peptides may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The peptides or mixture of peptides may be provided in particles. Particles as used herein means nanoparticles or microparticles/microspheres (or in some instances larger particles) which can consist in whole or in part of the compound or the other therapeutic agent(s) as described herein. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The peptides or mixture of peptides also may be dispersed throughout the particles. The peptides or mixture of peptides also may be adsorbed into the particles. The particles may be of any order release kinetics, including zero-order release, first-order release, second-order release, delayed release, sustained release, immediate release, and any combination thereof, etc. The particle may include, in addition to the peptides or mixture of peptides, any of those materials routinely used in the art of pharmacy and medicine, including, but not limited to, erodible, nonerodable, biodegradable, or nonbiodegradable material or combinations thereof. The particles may be microcapsules which contain the compound in a solution or in a semi-solid state. The particles may be of virtually any shape.

Both non-biodegradable and biodegradable polymeric materials can be used in the manufacture of particles for delivering the peptides or mixture of peptides. Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and polycaprolactone.

A therapeutic compound (e.g. a peptide or mixture of peptides) or other therapeutic agent or mixtures thereof can be formulated in a carrier system. The carrier can be a colloidal system. The carrier or colloidal system can be a liposome, a phospholipid bilayer vehicle. In one embodiment, therapeutic compound or other therapeutic agent or mixtures thereof can be encapsulated in a liposome while maintaining integrity of the therapeutic compound or other therapeutic agent or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g. a peptide or mixture of peptides) or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or poly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compound (e.g. a peptide or mixture of peptides) or other therapeutic agent or mixtures thereof are prepared with carriers that will protect the therapeutic compound, other therapeutic agent or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g. a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient for at least 2 days. In some embodiments, the implant or depot formulation is constructed and arranged to deliver therapeutic or prophylactic levels of the active ingredient for at least 7 days. In some embodiments, the implant or depot formulation is constructed and arranged to deliver therapeutic or prophylactic levels of the active ingredient for at least 14 days. In some embodiments, the implant or depot formulation is constructed and arranged to deliver therapeutic or prophylactic levels of the active ingredient for at least 30 days. In some embodiments, the implant or depot formulation is constructed and arranged to deliver therapeutic or prophylactic levels of the active ingredient for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient for at least 180 days. In some embodiments, the implant or depot formulation is constructed and arranged to deliver therapeutic or prophylactic levels of the active ingredient for at least one year. In some embodiments, the implant or depot formulation is constructed and arranged to deliver therapeutic or prophylactic levels of the active ingredient for 15-30 days. In some embodiments, the implant or depot formulation is constructed and arranged to deliver therapeutic or prophylactic levels of the active ingredient for 30-60 days. In some embodiments, the implant or depot formulation is constructed and arranged to deliver therapeutic or prophylactic levels of the active ingredient for 60-90 days. In some embodiments, the implant or depot formulation is constructed and arranged to deliver therapeutic or prophylactic levels of the active ingredient for 90-120 days. In some embodiments, the implant or depot formulation is constructed and arranged to deliver therapeutic or prophylactic levels of the active ingredient for 120-180 days. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

In some embodiments, the depot formulation comprises the peptide, or mixture of peptides, encapsulated or otherwise disposed within silica microparticles such those described in WO2000/050349, WO2001/013924, WO2001/015751, WO2001/040556, WO2002/080977, WO2005/082781, WO2007/135224, WO2008/104635, WO2014/207304 and WO2017/068845, wherein the active pharmaceutical ingredient to be delivered is the peptide or mixture of peptides disclosed herein. In some embodiments, the depot formulation is a sustained release formulation such that it provides for gradual release of a peptide or peptides (e.g. the peptide of Formula A) over an extended period of time, and that preferably, although not necessarily, results in substantially constant blood levels of a drug over an extended time period. In some embodiments, the sustained release occurs over days, weeks or months. In some embodiments, the sustained release occurs over a month or months, such as 1-2 months, 2-4 months, 3-5 months, 3-6 months, 5-7 months, 6-8 months, 6-9 months or 8-12 months.

It will be understood by one of ordinary skill in the relevant arts that other suitable modifications and adaptations to the compositions and methods described herein are readily apparent from the description of the present technology contained herein in view of information known to the ordinarily skilled artisan, and may be made without departing from the scope of the present application or any embodiment thereof.

### Determination of the Biological Effect of the Peptides or Mixtures of Peptides

In various embodiments, suitable in vitro or in vivo assays are performed to determine the effect of a peptide or mixture of peptides and whether its/their administration is indicated for treatment. In various embodiments, in vitro assays can be performed with representative animal models, to determine if a given peptide or mixture of peptides exerts the desired effect on the heart disease or cardiomyopathy of the subject. Compounds (e.g. a peptide or mixture of peptides) for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Animal models of DMD are known in the art, including, for example Golden retriever muscular dystrophy (GRMD) dogs, CXMDJ beagle dogs, hypertrophic feline muscular dystrophy (hfmd) cats, and mdx mice. See: Spurney C., Muscle Nerve 44(1):8-19 (2011); Willmann R. et al., Neuromuscular Disorders 19:241-249 (2009); Partridge TA, FEBS J. 280(17):4177-86 (2013) and Coley et al., “Effect of genetic background on the dystrophic phenotype in mdx mice”, Human Molecular Genetics, 2016, Vol. 25, No. 1, 130-145. More recently a rabbit model has been created that exhibits a very similar cardiac pathology to that observed in humans. See: Sui, T, et al., “A novel rabbit model of Duchenne muscular dystrophy generated by CRISPR/Cas9”, Disease Models & Mechanisms (2018) 11, dmm032201. Such models may be used to demonstrate the biological effect of the peptides and mixtures of peptides disclosed herein on the onset, incidence, severity and progression of heart disease and cardiomyopathies associated with MD (including DMD and BMD) in subjects, including humans.

### Combination Therapy

In some embodiments, the peptide or mixtures of peptides disclosed herein, may be combined with one or more additional therapies related to the treatment of (including without limitation the inhibition of, prevention of, amelioration of, or delaying the onset of) signs, symptoms, or severity of MD, DMD, or BMD in a subject, including a human subject. Additional therapeutic agents include, but are not limited to, corticosteroids, ACE inhibitors, ARB(s), beta-blockers, diuretics, angiotensin receptor blockers (ARBs), idebenone, phosphorodiamidate morpholino oligomers (PMOs). In some embodiments, the phosphorodiamidate morpholino oligomers comprise Exondys 51® (Eteplirsen), Golodirsen (Vyondys 53™)) or Casimersen (Amondys 45™) or a PPMO.

In some embodiments, the corticosteroids are selected from the group consisting of prednisone and deflazacort. In some embodiments, the ACE inhibitors are selected from the group consisting of captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, randolapril, and pharmaceutically acceptable salts of such compounds. In some embodiments, the ARBs are selected from the group consisting of losartan, candesartan, valsartan, eprosartan, telmisartan, and irbesartan.

In some embodiments, when an additional therapeutic agent is administered to a subject in combination with the peptide or mixture of peptides, a synergistic therapeutic effect is produced. For example, administration of the peptide or mixture of peptides with one or more additional therapeutic agents for addressing the signs, symptoms, or severity of muscular dystrophy (e.g. DMD or BMD) will have greater than additive effects in the treatment of the disease. For example, lower doses of one or more of any individual therapeutic agent may be used in treating or preventing DMD, resulting in increased therapeutic efficacy and decreased side-effects. Alternatively, for example, higher doses of one or more of: (i) corticosteroids, (ii) ACE inhibitors, (iii) ARB(s), (iv) beta-blockers, (v) diuretics, (vi) idebenone, and/or (vii) phosphorodiamidate morpholino oligomers (e.g. Exondys 51® (Eteplirsen), Golodirsen (Vyondys 53™)) or Casimersen (Amondys 45™)) may be used than is otherwise tolerable because treatment with the peptide or mixture of peptides described herein protects the subject from detrimental effects that otherwise affect the subject’s heart. In some embodiments, the synergistic effect will be improved ambulation (or delay in reduction in ambulation) resulting from the combined effects of increases in muscular dystrophin with increases in muscle function and energy associated with improved mitochondrial health of the subject (and the subject’s muscles) in combination with delayed, decreased, ameliorated or inhibited cardiovascular stress and associated cardiomyopathy (e.g. HCM, DCM, heart failure and/or cardiac fibrosis).

In any case, the multiple therapeutic agents (e.g. a peptide (e.g. Compound A-1 or A-2) or mixture of peptides (e.g. Compound A-1 and A-2) in combination with Exondys 51® (Eteplirsen), Golodirsen (Vyondys 53™)) or Casimersen (Amondys 45™) may be administered in any order or even simultaneously. If simultaneously, the multiple therapeutic agents may be provided in a single, unified form, or in multiple forms (by way of example only, either as an IV injection or as two separate IV injections). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Anticipated Therapeutic Effects

In certain embodiments, DMD subjects treated with the peptide or peptide mixtures will show normalization of creatine phosphokinase blood levels by at least 5%, at least 10%, at least 25%, at least 50%, at least 75%, or at least 90% compared to untreated DMD subjects. In certain embodiments, DMD subjects treated with the peptide or peptide mixtures will show creatine phosphokinase blood levels that are similar to that observed in a normal control subject.

In some embodiments of the methods, DMD subjects treated with the peptide or peptide mixtures will show an increase in utrophin expression levels and/or activity by at least 5%, at least 10%, at least 25%, at least 50%, at least 75%, or at least 90% compared to an untreated DMD control subject. In certain embodiments, DMD subjects treated with the peptide or peptide mixtures will show an increase in IGF-1 expression levels and/or activity by at least 5%, at least 10%, at least 25%, at least 50%, at least 75%, or at least 90% compared to an untreated DMD control subject. In some embodiments of the methods, DMD subjects treated with the peptide or peptide mixtures will show an increase in follistatin expression levels and/or activity by at least 5%, at least 10%, at least 25%, at least 50%, at least 75%, or at least 90% compared to an untreated DMD control subject.

In some embodiments of the methods, DMD subjects treated with the peptide or peptide mixtures will show improvements in cardiac function as compared with untreated control group subjects. For subjects treated with both the peptide or peptide mixtures and a drug that increases dystrophin expression, improvements in cardiac function and ambulation would be expected as compared with untreated control group subjects.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The present technology is further illustrated by the following examples, which should not be construed as limiting in any way.

### Example 1- Use of H-D-Arg-2′6′-Dmt-Lys-Phe-NH₂ in the Treatment of Cardiomyopathy Progression in a DMD Knock Out (KO) Rabbit Model

This Example prophetically demonstrates the use of H-D-Arg-2′6′-Dmt-Lys-Phe-NH₂ (elamipretide), in the treatment of cardiomyopathy progression (as well as other symptoms & physiology commonly observed in DMD patients) in a DMD KO rabbit model.

Rabbits: The DMD KO rabbits used in this study will be obtained from the Laboratory Animal Center of Jilin University or prepared as described in Sui et al., Disease Models & Mechanisms 11 (2018). These DMD KO rabbits possess an engineered mutation in exon 51. These DMD KO rabbits exhibit several of the human characteristics of DMD pathology, including disruption of dystrophin expression, impaired physical activity (loss of ambulation), lower body mass, shorter lifespan, elevated serum creatine kinase (CK) levels, and most significantly, a progressive cardiomyopathy leading to heart failure similar to that observed in humans. New Zealand rabbits (WT) will be used as controls.

Methods: All experiments involving rabbits in this study will be performed in accordance with all applicable laws and regulations.

Body Weight & Survival Curve: The body weight of age- and sex-matched WT and DMD KO rabbits will be measured biweekly. All data will be expressed as the mean±s.e.m., and a minimum of three individual animals of each genotype will be used in all experiments.

Serum Biochemical Analysis: The blood samples will be collected into heparinized tubes from the ear vein, and sera will be prepared by precipitation and centrifugation. Serum CK, alanine aminotransferase (ALT) and aspartate aminotransferase (AST) levels will be measured using a CK test kit (N-acetyl-L-cysteine method), ALT test kit (continuous monitoring method) and AST test kit (continuous monitoring method), respectively.

Activity Measurement: A Millet Sports Bracelet Wearable Device (or other suitable device) will be used to record movement steps within a 1-hour period for DMD KO and WT rabbits. The rabbits wearing the device on their right hind leg will be placed in an appropriately sized room to allow for free movement.

Echocardiology: Echocardiography recording will be performed as described previously (Han et al., 2007; Xu et al., 2015a). Briefly, two-dimensional and M-mode transthoracic echocardiography will be performed as previously described on WT and DMD KO rabbits (n≥3 per group) by an SIUI all digital color doppler ultrasound diagnostic system such as an Apogee 300, ShanTou, China. Rabbits will be studied in right lateral recumbency from parasternal long and short axis views. The rabbits will be held in the right position by restraining their limbs with people. A linear array probe and center frequency of 10.0 MHz will be used. Cardiac dimensions [the interventricular septal thickness at end-diastole (IVSd), left ventricular end diastolic diameter (LVDd) and left ventricular systolic diameter (LVDs)] will be determined and the percentage of fractional shortening (FS) and left ventricular ejection fraction (EF) will be calculated. If possible, stroke volume and cardiac output will also be assessed.

Histology: Cardiac tissue will be collected from DMD KO and WT rabbits (euthanized at 5-6 and 10-12 months of age). The tissues will be fixed in 4% paraformaldehyde at 4° C., dehydrated in increasing concentrations of ethanol (70% for 6 hours, 80% for 1 hour, 96% for 1 hour and 100% for 3 hour), cleared in xylene and embedded in paraffin for histological examination. The 5-µm sections will be cut for H&E (Han et al., 2007; Xu et al., 2015a) to investigate monocytic infiltrates/inflammation. Fibrosis of cardiac muscle will be assessed with Masson’s Trichrome staining and anti-collagen immunohistochemistry using standard practices. The stained sections will be imaged with an appropriate microscope such as a Nikon TS100 microscope.

Study Design: At approximately 3-4 weeks of age, rabbits will be administered peptide (or a mixture of peptides) dissolved in sterile saline in dose(s) ranging from 0.5 to 5 mg/kg, once daily via intraperitoneal (i.p.) injection. The control group will be given placebo (sterile saline). Rabbits will be treated daily with peptide (or a mixture of peptides) or saline vehicle control, with treatment lasting approximately 3-5 months. For example, the peptide used could be a peptide of Formula A-1 (i.e. H-D-Arg-2′6′-Dmt-Lys-Phe-NH₂). Echocardiograms will be collected monthly until animals are approximately 4-6 months of age or end of life. At the time of death, tissues will be harvested and variables described above will be collected.

### Anticipated Results

1. Weight and Survival: It is anticipated that the DMD KO rabbits will be smaller in size and have a significantly shorter lifespan as compared with the WT rabbit. However, it is anticipated that the DMD KO rabbits that are treated with a peptide of Formula A (e.g. Compound A-1, A-2, A-3, A-4, A-5, A-6, A-7 or A-8) will have a statistically significant increase in lifespan as compared with the untreated control group of DMD KO rabbits, which increase in lifespan will be attributable to delay in onset and progression of the cardiomyopathies and resulting heart failure.

2. Serum biochemistry analysis: It is anticipated that the DMD KO rabbits will exhibit a significant increase in serum CK, alanine aminotransferase (ALT) and aspartate aminotransferase (AST) levels as compared with WT rabbits. However, it is anticipated that the levels of serum CK, ALT and AST will be statistically lower (and closer to those levels seen in the WT rabbits) in the DMD KO rabbits treated with a peptide of Formula A (e.g. Compound A-1, A-2, A-3, A-4, A-5, A-6, A-7 or A-8) as compared with the DMD KO rabbits that are not treated with the peptide.

3. Activity measurement: It is anticipated that the DMD KO rabbits will exhibit a significant decrease in activity as compared with WT rabbits. However, it is anticipated that the levels of levels of activity will increase in the DMD KO rabbits treated with a peptide of Formula A (e.g. Compound A-1, A-2, A-3, A-4, A-5, A-6, A-7 or A-8) as compared with the DMD KO rabbits that are not treated with the peptide.

4. Histology of muscles: It is expected that cardiac muscle of the DMD KO rabbits will exhibit a lower level of inflammation and fibrosis in the treated group as compared with the untreated group.

5. Echocardiography: It is anticipated that the DMD KO rabbits will exhibit a significant decrease left ventricular ejection fraction (EF) and fractional shortening (FS) as compared with WT rabbits. However, it is anticipated that the levels of left ventricular ejection fraction (EF) and fractional shortening (FS) will be higher in the DMD KO rabbits treated with a peptide of Formula A (e.g. Compound A-1, A-2, A-3, A-4, A-5, A-6, A-7 or A-8) as compared with the DMD KO rabbits that are not treated with the peptide. Further, it is anticipated that the treated rabbits will exhibit a delay in the onset of the decrease in levels of left ventricular ejection fraction (EF) and fractional shortening (FS) as compared with untreated rabbits. If data on stroke volume and cardiac output are obtained, it is expected that treated rabbits will exhibit higher stroke volume and improved cardiac output as compared with untreated DMD KO rabbits.

Accordingly, these results will demonstrate that the peptides of the present technology, such as H-D-Arg-2′6′-Dmt-Lys-Phe-NH₂ (elamipretide), are useful in methods for treating or delaying the onset of cardiomyopathy (e.g. hypertrophic cardiomyopathy, dilated cardiomyopathy, heart failure or cardiac fibrosis) in a mammalian subject suffering from muscular dystrophy, such as Duchenne muscular dystrophy (DMD) or Becker muscular dystrophy (BMD).

### Example 2 - Use of H-D-Arg-2′6′-Dmt-Lys-Phe-NH₂ in the Treatment of Cardiomyopathy in a D2-mdx Mouse Model

This Example prophetically demonstrates the use of H-D-Arg-2′6′-Dmt-Lys-Phe-NH₂ (elamipretide), in the treatment of cardiomyopathy progression (as well as other symptoms & physiology commonly observed in DMD patients) in a DMD mouse model that exhibits traits of the hypertrophic cardiomyopathy observed in humans afflicted with muscular dystrophy.

Animals and Care: The DBA2 (a.k.a. D2-mdx) congenic strain of mdx mouse will be used for these studies. DBA/2J mice will be used as the wildtype control group. Animals will be obtained from appropriate sources (e.g. Jackson Labs). Animals will be housed at a density of up to five males or five females per cage under specific pathogen-free conditions in rooms with a 12-h light/12-h dark cycle at a temperature of 18-23° C. and 40-60% humidity. All experiments involving mice in this study will be performed in accordance with all applicable laws and regulations.

Genotyping: Genotyping of the animals will be performed according to the Reference (Coley et al.). The DBA2 mouse strain has been reported to have a mutation in the LTBP4 gene that may affect muscle function and regeneration. Briefly, genotyping for this reported deletion mutation will be carried out by standard PCR, followed by gel electrophoresis. The product size of the wild-type LTBP4 band is 273 bp, whereas the band for the mutant is only 236 bp. Genotyping of the MDX exon 23 SNP will be performed via real-time allelic discrimination using custom made Taqman® fluorescent-bound primers, reagents and protocol as described in the Reference Coley et al.).

Voluntary Wheel Running: Animals will be placed in individual cages equipped with a locked 14 cm diameter running wheel and rotation counter. Following a 24 hour acclimation period, the wheel will be unlocked and the distance run over 24 hours will be recorded. Recordings will be performed weekly throughout the duration of the experiment.

Serum creatine kinase activity: Blood will be collected from mice via cardiac puncture immediately following sacrifice. Serum will be separated from other blood fractions by centrifugation and stored at -80° C. without EDTA or heparin. CK activity in the serum will be measured using an appropriate commercially available product (e.g. the Creatine Kinase Reagent Set from Pointe Scientific, Inc. (Canton, MI)) according to the manufacturer’s protocol. Alternatively, blood can be collected by retro-orbital sinus puncture into heparinized glass capillaries. Serum can be isolated by centrifugation for 10 min at 19,000 rpm. Creatine kinase can be measured, for example, with a Beckman Coulter AU Clinical Chemistry analyzer

Echocardiography: In order to assess the cardiac function of mice in vivo, echocardiography will be performed on sedated mice. Mice will first be anesthetized (e.g. with 5% isoflurane mixed with 100% oxygen at 1.0 1/min flow) and then maintained under anesthesia (e.g. with 1.5% isoflurane/oxygen flow. Ophthalmic ointment can be placed on the eyes of the animals to prevent drying of the cornea while the mouse was anesthetized and tested. After anesthetic induction, the animals will be placed on a thermostatically controlled heated platform, where anesthesia can be maintained by delivery through a close-fitting facemask. A heating lamp can also be used to keep the heart rate and body temperature constant at physiological status during echocardiography.

During the examination, the animal’s heart rate will be monitored through the use of an electrocardiograph. The mouse’s heart rate and body temperature will be monitored continuously during the scanning. Echocardiography will be performed using an appropriate apparatus (e.g. Vevo770 ultrasound machine (VisualSonics, Toronto, Canada). The 2-D (B-mode), M-mode and Doppler images will be acquired from a modified parasternal long axis view, parasternal short axis view, suprasternal notch view and apical three-chamber view. The heart rate (BPM), fractional shortening, EF, stroke volume and cardiac output will be obtained for cardiac function assessment using measurements from the modified parasternal short axis view in the M-mode. Qualitative and quantitative measurements will be recorded using offline workstation software, and post-imaging analysis will be performed.

Histology: Cardiac tissue will be collected from DBA2 and DBA/2J mice (euthanized at 28 weeks of age). The tissues will be fixed in 4% paraformaldehyde at 4° C., dehydrated in increasing concentrations of ethanol (70% for 6 hours, 80% for 1 hour, 96% for 1 hour and 100% for 3 hour), cleared in xylene and embedded in paraffin for histological examination. 5-µm sections will be cut for H&E (Han et al., 2007; Xu et al., 2015a) to investigate monocytic infiltrates/inflammation. Fibrosis of cardiac muscle will be assessed with Masson’s Trichrome staining and anti-collagen immunohistochemistry using standard practices. Tissue sections will be imaged using an appropriate camera apparatus (e.g. Olympus BX51 microscope with attached Olympus DP70 camera module).

### Mouse Strains & Study Design:

Strains (Commercially Available from Jackson Labs):
DBA/2J (stock number: 000671) - wildtype control
D2-mdx (stock number: 013141) - dystrophic

Study Design
1. N=40-60 mice total assigned to following groups
   a. Wildtype control - untreated, n=10-15
   b. mdx, vehicle treated, n=10-15
   c. mdx, daily IP elamipretide 1.0 mg/kg, n=10-15
   d. mdx, daily IP elamipretide 5.0 mg/kg, n=10-15
2. Dosing to begin at 4-6 weeks of age and continue for 10-15 weeks
   a. Weekly retroorbital eye bleeds will be taken to provide serum for biomarker analysis
3. In-life endpoints
   a. Weekly body weight will be taken
   b. Measurements of weekly voluntary wheel running will be determined
      i. Total distance (m)
      ii. Normalized distance (m/kg)
   c. Echocardiogram (at least at week 18 & end of study but possibly more often, including possibly bi-monthly). The following parameters will be accessed.
      i. Ejection fraction (%)
      ii. Fractional shortening (%)
      iii. Stroke volume (uL)
      iv. Cardiac output (mL)
      v. Heart rate (bpm)
4. Endpoints post-necropsy
   a. Serum analysis for creatine kinase
   b. Histology of heart and inflammation and fibrosis

### Anticipated Results:

1. Weight and Survival: It is anticipated that the D2-mdx mice will be smaller in size as compared with the DBA/2J (control) mice. However, it is anticipated that the D2-mdx mice that are treated with a peptide of Formula A (e.g. Compound A-1, A-2, A-3, A-4, A-5, A-6, A-7 or A-8) will display accelerated weight gain relative to the untreated control group.

2. Serum biochemistry analysis: It is anticipated that the D2-mdx mice will exhibit a significant increase in serum CK levels as compared with DBA/2J (control) mice. However, it is anticipated that the levels of serum CK will be statistically lower (and closer to those levels seen in the DBA/2J (control) mice) in the D2-mdx mice treated with a peptide of Formula A (e.g. Compound A-1, A-2, A-3, A-4, A-5, A-6, A-7 or A-8) as compared with the D2-mdx mice that are not treated with the peptide.

3. Voluntary Wheel Running: It is anticipated that the D2-mdx mice will exhibit a significant decrease in total and normalized distance run per 24 hours as compared with DBA/2J (control) mice. However, it is anticipated that the total and normalized distance run will increase in the D2-mdx mice treated with a peptide of Formula A (e.g. Compound A-1, A-2, A-3, A-4, A-5, A-6, A-7 or A-8) as compared with the D2-mdx mice that are not treated with the peptide.

4. Histology of heart: It is anticipated that the D2-mdx mice will display significantly increased levels of inflammation (as measured by H+E stain for mononuclear cellular infiltrates) and fibrosis (as measured by Masson’s Trichrome stain and anti-collagen immunohistochemistry) relative to DBA/2J control mice. However, it is anticipated that inflammation and fibrosis will decrease in the D2-mdx mice treated with a peptide of Formula A (e.g. Compound A-1, A-2, A-3, A-4, A-5, A-6, A-7 or A-8) as compared with the D2-mdx mice that are not treated with the peptide.

5. Echocardiography: It is anticipated that the D2-mdx mice will exhibit a significant decrease in left ventricular ejection fraction (EF), fractional shortening (FS), stroke volume and cardiac output as compared with DBA/2J (control) mice. However, it is anticipated that the levels of left ventricular ejection fraction (EF), fractional shortening (FS), stroke volume and cardiac output will be higher in the D2-mdx mice treated with a peptide of Formula A (e.g. Compound A-1, A-2, A-3, A-4, A-5, A-6, A-7 or A-8) as compared with the D2-mdx mice that are not treated with the peptide. Further, it is anticipated that the treated mice will exhibit a delay in the onset of the decrease in levels of left ventricular ejection fraction (EF), fractional shortening (FS), stroke volume and cardiac output as compared with untreated mice.

Accordingly, these results will demonstrate that the peptides of the present technology, such as H-D-Arg-2′6′-Dmt-Lys-Phe-NH2 (elamipretide), are useful in methods for treating or delaying the onset of cardiomyopathy (e.g. hypertrophic cardiomyopathy, dilated cardiomyopathy, heart failure or cardiac fibrosis) in a mammalian subject suffering from muscular dystrophy, such as Duchenne muscular dystrophy (DMD) or Becker muscular dystrophy (BMD).

### Example 3 - Use of H-D-Arg-2′6′-Dmt-Lys-Phe-NH₂ or H-D-Arg-2′6′-Dmt-Lys-Phe-OH in the Treatment of Cardiomyopathies in Human Subjects Diagnosed with Muscular Dystrophy

This example prophetically demonstrates the use of H-D-Arg-2′6′-Dmt-Lys-Phe-NH₂ or H-D-Arg-2′6′-Dmt-Lys-Phe-OH in treating or delaying the onset of cardiomyopathy in a human subject in need thereof who has been diagnosed with muscular dystrophy, such as Duchenne muscular dystrophy (DMD) or Becker muscular dystrophy (BMD).

### Methods

Subjects suspected of having or diagnosed as having DMD or BMD receive daily subcutaneous administration of H-D-Arg-2′6′-Dmt-Lys-Phe-NH₂ or H-D-Arg-2′6′-Dmt-Lys-Phe-OH (e.g. with 0.5-5.0 mg/kg/day). Alternatively, subjects suspected of having or diagnosed as having DMD or BMD receive weekly intravenous administration of H-D-Arg-2′6′-Dmt-Lys-Phe-NH₂ or H-D-Arg-2′6′-Dmt-Lys-Phe-OH (e.g. with 0.05-1.0 mg/kg/hr. for up to 4 hours). In some cases, the subjects could be co-administered a drug known to increase or correct the production of dystrophin in the subject, such as a phosphorodiamidate morpholino oligomer (PMO) such as Eteplirsen (Exondys 51®), Golodirsen (Vyondys 53™) or Casimersen (Amondys 45™) or a PPMO. Subjects will be regularly evaluated (e.g., weekly, bi-weekly, monthly, etc.) for the presence and/or severity of signs and symptoms of cardiomyopathy associated with DMD or BMD including, but not limited to, impaired left ventricular dynamics, e.g., reduced ejection fraction, reduced shortening fraction, reduced stroke volume, and/or reduced cardiac output, and elevated levels serum biomarkers associated with myocardial fibrosis, such as, e.g., elevated levels of carboxy terminal peptide of procollagen type I (PICP) and/or amino terminal propeptide of procollagen type III. Treatments will be maintained at least until such a time as one or more signs or symptoms of cardiomyopathy associated with DMD or BMD are ameliorated or eliminated. The study may be conducted in a randomized withdrawal trial (e.g. randomized, double-blind, placebo-controlled withdrawal trial) to assess the impact of the peptide on the subjects followed by the impact of its withdrawal relative to a control group still receiving the peptide (and/or a drug known to increase or correct the production of dystrophin in the subject).

### Results

It is predicted that subjects suspected of having or diagnosed as having DMD or BMD and receiving therapeutically effective amounts of H-D-Arg-2′6′-Dmt-Lys-Phe-NH₂ or H-D-Arg-2′6′-Dmt-Lys-Phe-OH will display reduced severity or elimination of one or more signs or symptoms of cardiomyopathy associated with DMD or BMD. These results will show that H-D-Arg-2′6′-Dmt-Lys-Phe-NH₂ or H-D-Arg-2′6′-Dmt-Lys-Phe-OH is useful in ameliorating one or more of the following symptoms of cardiomyopathy associated with DMD or BMD: impaired left ventricular dynamics, e.g., reduced ejection fraction, reduced shortening fraction, reduced stroke volume, and/or reduced cardiac output, and elevated levels serum biomarkers associated with myocardial fibrosis, such as, e.g., elevated levels of carboxy terminal peptide of procollagen type I (PICP) and/or amino terminal propeptide of procollagen type III as compared to untreated controls. Accordingly, these results will demonstrate that H-D-Arg-2′6′-Dmt-Lys-Phe-NH₂ or H-D-Arg-2′6′-Dmt-Lys-Phe-OH is useful in methods for treating or delaying the onset of cardiomyopathy in subjects suspected of having or diagnosed as having DMD or BMD.

Other signs or symptoms of DMD that might be evaluated and result in improvements include:
Delay in the progression of left ventricle dilation
Delay in the progression of left ventricle fibrosis
Delay in the reduction of left ventricle stroke volume
Improvements in left ventricle end diastolic volume (LVEDV)
Improvements in left ventricle end systolic volume (LVESV)
Change in myocardial strain
Improvements in respiratory function
Improvements in the subject’s ambulation (e.g. delay in onset or progression of the subject’s decline in ambulation or outright improvement in the subject’s ability to move)

### Example 4 - Use of H-D-Arg-2′6′-Dmt-Lys-Phe-NH₂ or H-D-Arg-2′6′-Dmt-Lys-Phe-OH in the Treatment of Cardiomyopathies in Human Subjects Diagnosed with DMD and Taking Corticosteroids and/or Eteplirsen (Exondys 51®), Golodirsen (Vyondys 53™)or Casimersen (Amondys 45™)

This example prophetically demonstrates the use of H-D-Arg-2′6′-Dmt-Lys-Phe-NH₂ or H-D-Arg-2′6′-Dmt-Lys-Phe-OH in treating or delaying the onset of cardiomyopathy in a subject in need thereof who has been diagnosed with muscular dystrophy, such as Duchenne muscular dystrophy (DMD) or Becker muscular dystrophy (BMD), and taking corticosteroids and/or Eteplirsen (Exondys 51®), Golodirsen (Vyondys 53™)or Casimersen (Amondys 45™).

### Methods

Subjects suspected of having or diagnosed as having DMD or BMD and taking corticosteroids and/or Eteplirsen (Exondys 51®), Golodirsen (Vyondys 53™)or Casimersen (Amondys 45™) are separately, sequentially, or simultaneously subcutaneously administered a daily dose of H-D-Arg-2′6′-Dmt-Lys-Phe-NH₂ or H-D-Arg-2′6′-Dmt-Lys-Phe-OH (e.g. with 0.5-5.0 mg/kg/day) or a weekly intravenous administration of H-D-Arg-2′6′-Dmt-Lys-Phe-NH₂ or H-D-Arg-2′6′-Dmt-Lys-Phe-OH (e.g. with 0.05-1.0 mg/kg/hr. for up to 4 hours). Subjects will be regularly evaluated (e.g.,, weekly, bi-weekly, monthly, etc.) for the presence and/or severity of signs and symptoms of cardiomyopathy associated with DMD or BMD including, but not limited to, impaired left ventricular dynamics, e.g., reduced ejection fraction, reduced shortening fraction, reduced stroke volume, and/or reduced cardiac output, and elevated levels serum biomarkers associated with myocardial fibrosis, such as, e.g., elevated levels of carboxy terminal peptide of procollagen type I (PICP) and/or amino terminal propeptide of procollagen type III. Treatments are maintained at least until such a time as one or more signs or symptoms of cardiomyopathy associated with DMD or BMD are ameliorated or eliminated. The study may be conducted in a randomized withdrawal trial (e.g. randomized, double-blind, placebo-controlled withdrawal trial) to assess the impact of the peptide on the subjects followed by the impact of its withdrawal relative to a control group still receiving the peptide.

### Results

It is predicted that subjects suspected of having or diagnosed as having DMD or BMD and taking corticosteroids and/or Eteplirsen (Exondys 51®), Golodirsen (Vyondys 53™)or Casimersen (Amondys 45™) and receiving therapeutically effective amounts of H-D-Arg-2′6′-Dmt-Lys-Phe-NH₂ or H-D-Arg-2′6′-Dmt-Lys-Phe-OH will display delayed or reduced severity or elimination of one or more signs or symptoms of cardiomyopathy associated with DMD or BMD. It is further expected that administration of H-D-Arg-2′6′-Dmt-Lys-Phe-NH₂ or H-D-Arg-2′6′-Dmt-Lys-Phe-OH in combination with corticosteroids and/or Eteplirsen (Exondys 51®), Golodirsen (Vyondys 53™)or Casimersen (Amondys 45™) will have synergistic effects in this regard compared to that observed in subjects treated with H-D-Arg-2′6′-Dmt-Lys-Phe-NH₂ or H-D-Arg-2′6′-Dmt-Lys-Phe-OH alone, or corticosteroids and/or Eteplirsen (Exondys 51®), Golodirsen (Vyondys 53™)or Casimersen (Amondys 45™) alone.

These results will show that H-D-Arg-2′6′-Dmt-Lys-Phe-NH₂ or H-D-Arg-2′6′-Dmt-Lys-Phe-OH is useful in ameliorating one or more of the following symptoms of cardiomyopathy associated with DMD or BMD such as impaired left ventricular dynamics (e.g., reduced ejection fraction, reduced shortening fraction, reduced stroke volume, and/or reduced cardiac output), and elevated levels serum biomarkers associated with myocardial fibrosis, such as, e.g., elevated levels of carboxy terminal peptide of procollagen type I (PICP) and/or amino terminal propeptide of procollagen type III as compared to untreated controls. Accordingly, these results will demonstrate that H-D-Arg-2′6′-Dmt-Lys-Phe-NH₂ or H-D-Arg-2′6′-Dmt-Lys-Phe-OH is useful in methods for treating or delaying the onset of cardiomyopathy in subjects suspected of having or diagnosed as having DMD or BMD and taking corticosteroids and/or Eteplirsen (Exondys 51®), Golodirsen (Vyondys 53™)or Casimersen (Amondys 45™).

Other signs or symptoms of DMD that might be evaluated and improvements in observed include:
Delay in the progression of left ventricle dilation
Delay in the progression of left ventricle fibrosis
Delay in the reduction of left ventricle stroke volume
Improvements in left ventricle end diastolic volume (LVEDV)
Improvements in left ventricle end systolic volume (LVESV)
Change in myocardial strain
Improvements in respiratory function
Improvements in the subject’s ambulation (e.g. delay in onset or progression of the subject’s decline in ambulation or outright improvement in the subject’s ability to move)

### Example 5 - One-Step Synthesis of 1-((R)-4-ammonio-5-(((S)-1-(((S)-6-ammonio-1-(Y(S)-1-carboxy-2-phenylethyl)amino)-1-oxohexan-2-yl)amino)-3-(4-hydroxy-2,6-dimethylphenyl)-1-oxopropan-2-yl)amino)-5-oxopentyl)guanidinium Chloride (A-2, Tris-HCl salt) From Elamipretide (A-1, Tris Acetate Salt)

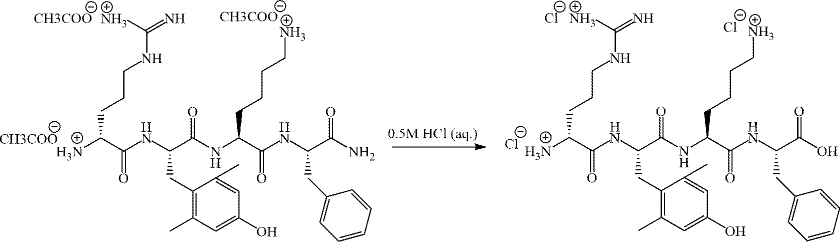

A solution of SBT-031 triacetate (8.2 g, 10 mmol) in 0.5 M aq. hydrochloric acid was stirred at 35-40° C. for 5 days. Then solvent was removed under reduced pressure and crude product purified by reversed phase flash chromatography (water (pH=3)/MeCN, from 0.25 to 4%) to give A-2 (5.6 g) in 75% yield.

¹H-NMR (400 MHz, Methanol-d4) δ 7.37 - 7.14 (m, 5H), 6.43 (s, 2H), 4.80 (dd, J = 9.5, 6.9 Hz, 1H), 4.64 (dd, J = 8.6, 5.2 Hz, 1H), 4.37 (dd, J = 8.1, 5.9 Hz, 1H), 4.01 (t, J = 6.3 Hz, 1H), 3.21 (dd, J = 14.0, 5.2 Hz, 1H), 3.16 - 2.88 (m, 7H), 2.27 (s, 6H), 1.85 - 1.54 (m, 6H), 1.53 - 1.20 (m, 4H).

### EQUIVALENTS

The present technology is not to be limited in terms of the particular embodiments described herein, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Various embodiments are set forth within the following claims.

What is claimed is:

1. A method for treating cardiomyopathy or delaying the onset of cardiomyopathy in a mammalian subject suffering from muscular dystrophy, comprising administering to the subject a therapeutically effective amount of a peptide of formula A: 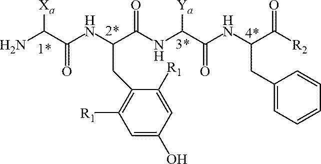
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 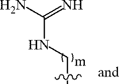 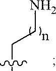
each m is 2, 3 or 4; each n is independently 1, 2, or 3; and the absolute stereochemistry at each of stereocenters 1*, 2*, 3*, and 4* is independently D or L.

2. The method of claim 1, wherein the peptide of generic Formula A is a peptide of formula A-1: 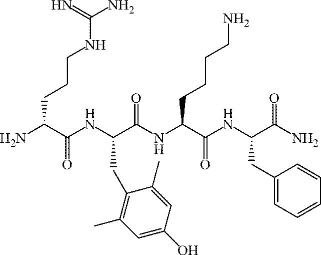 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]
or a pharmaceutically acceptable salt, hydrate, solvate and/or tautomer thereof.

3. The method of claim 1, wherein the peptide of generic Formula A is a peptide of formula A-2: 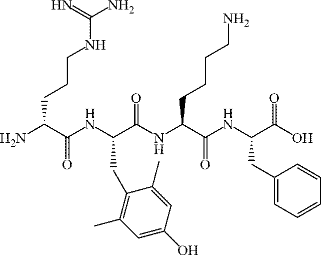 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]
or a pharmaceutically acceptable salt, hydrate, solvate and/or tautomer thereof.

4. The method of claim 1, wherein the peptide of generic Formula A is a peptide of formula A-3, A-4, A-5, A-6, A-7 or A-8: 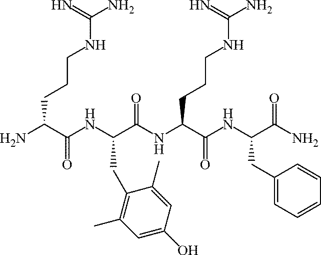 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O] 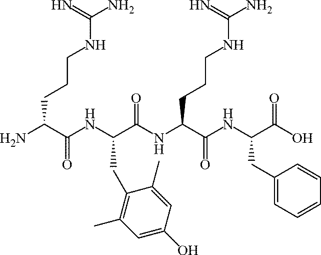 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O] 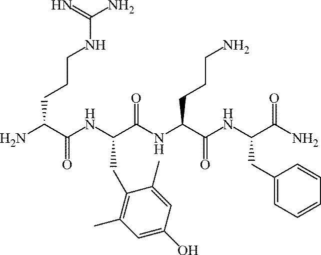 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O] 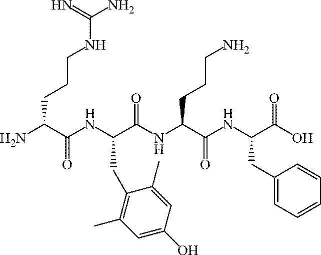 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O] 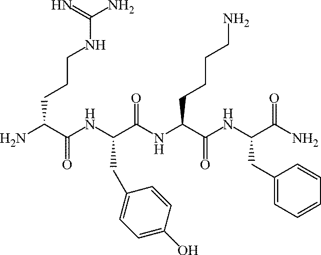 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O] 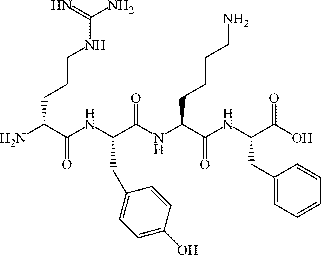 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]
or a pharmaceutically acceptable salt, hydrate, solvate, and/or tautomer thereof.

5. The method of any one of claims 1 to 4, wherein administration of the peptide reduces, ameliorates, and/or delays the onset of hypertrophic cardiomyopathy, dilated cardiomyopathy, heart failure and/or cardiac fibrosis in a subject diagnosed with and/or being treated for muscular dystrophy.

6. The method of claim 5, wherein administration of the peptide prevents, inhibits, reduces, ameliorates, and/or delays the onset of hypertrophic cardiomyopathy.

7. The method of claim 5, wherein administration of the peptide prevents, inhibits, reduces, ameliorates, and/or delays the onset of dilated cardiomyopathy.

8. The method of claim 5, wherein administration of the peptide prevents, inhibits, reduces, ameliorates, and/or delays the onset of heart failure.

9. The method of claim 5, wherein administration of the peptide prevents, inhibits, reduces, ameliorates, and/or delays the onset of cardiac fibrosis.

10. The method of any one of claims 1 to 9, wherein administration of the peptide increases the ejection fraction, shortening fraction, stroke volume, or cardiac output of the heart of the subject as compared with the heart of an untreated control subject or control group that is not administered the peptide.

11. The method of any one of claims 1 to 10, wherein the peptide is administered daily for: (i) 24 weeks or more; (ii) 48 weeks or more; (iii) 72 weeks or more; or (iv) 96 weeks or more.

12. The method of any one of claims 1 to 10, wherein the peptide is administered once weekly for: (i) 24 weeks or more; (ii) 48 weeks or more; (iii) 72 weeks or more; or (iv) 96 weeks or more.

13. The method of any one of claims 1 to 12, wherein the peptide is administered orally, topically, systemically, intraperitoneally, intradermally, transdermally, ophthalmically, intrathecally, intracerebroventricularly, iontophoretically, transmucosally, intravitreally, intranasally, or intramuscularly.

14. The method of claim 11, wherein the peptide is administered subcutaneously.

15. The method of claim 12, wherein the peptide is administered intravenously.

16. The method of any one of claims 1 to 15, wherein the subject is human.

17. The method of any one of claims 1 to 16, wherein the muscular dystrophy is Duchenne muscular dystrophy (DMD).

18. The method of any one of claims 1 to 16, wherein the muscular dystrophy is Becker muscular dystrophy (BMD).

19. The method of any one of claims 1 to 18, further comprising separately, sequentially, or simultaneously administering an additional therapeutic agent to the subject.

20. The method of claim 19, wherein the peptide is administered to the subject in combination with a drug known to increase or correct the production of dystrophin in the subject.

21. The method of claim 20, wherein the subject has been diagnosed as having DMD and the peptide is administered to the subject in combination with a phosphorodiamidate morpholino oligomer (PMO), such as Eteplirsen (Exondys 51®), Golodirsen (Vyondys 53™) or Casimersen (Amondys 45™), or a PPMO.

22. The method of claim 21, wherein the peptide and the PMO or PPMO are administered intravenously.

23. The method of claim 22, wherein the peptide and the PMO or PPMO are administered simultaneously.

24. The method of claim 19, wherein the peptide is administered to the subject in combination with a corticosteroid.

25. The method of claim 19, wherein the peptide is administered to the subject in combination with an ACE inhibitor.

26. The method of claim 19, wherein the peptide is administered to the subject in combination with an ARB.

27. The method of claim 19, wherein the peptide is administered to the subject in combination with a beta blocker.

28. The method of any one of claims 19 to 27, wherein the combination of peptide and additional therapeutic agent has a synergistic effect in the treatment of DMD or BMD.

29. The method of any one of claims 1 to 28, wherein the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

30. The method of any one of claims 1 to 29, wherein the peptide of Formula A is administered in a depot formulation.

31. The method of claim 30, wherein the depot formulation comprises the peptide of Formula A encapsulated or otherwise disposed in silica microparticles.

32. The method of claims 30 or 31, wherein the depot formulation is a sustained release depot formulation.

33. The method of claim 32, wherein the peptide of Formula A is released in an effective amount over days, weeks or months.

34. Use of a composition in the preparation of a medicament for treating cardiomyopathy or delaying the onset of cardiomyopathy in a mammalian subject suffering from muscular dystrophy, wherein the composition comprises a therapeutically effective amount of a peptide of formula A: 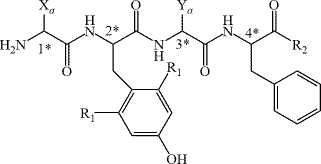
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 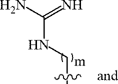 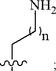
each m is 2, 3 or 4; each n is independently 1, 2, or 3; and the absolute stereochemistry at each of stereocenters 1*, 2*, 3*, and 4* is independently D or L.

35. The use of claim 34, wherein the peptide of generic Formula A is a peptide of formula A-1 or A-2: 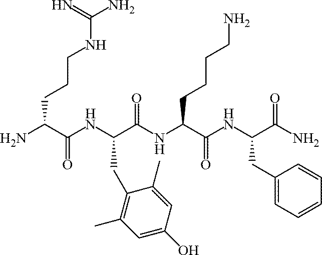 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O] 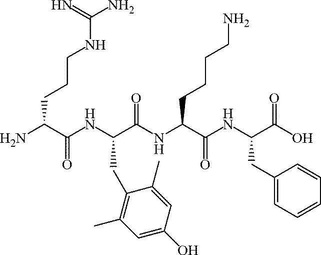 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]
or a pharmaceutically acceptable salt, hydrate, solvate and/or tautomer thereof.

36. The use of claim 34, wherein the peptide of generic Formula A is a peptide of formula A-3, A-4, A-5, A-6, A-7 or A-8: 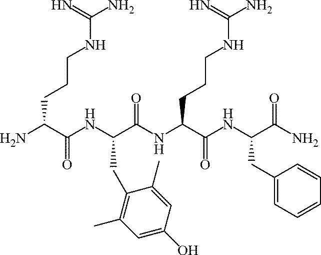 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(N)=O] 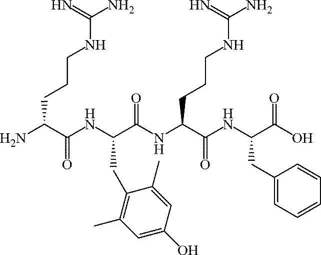 [SMILES: Cc1cc(O)cc(C)c1C[C@H](NC(=O)[C@H](N)CCCNC(=N)N)C(=O)N[C@@H](CCCNC(=N)N)C(=O)N[C@@H](Cc1ccccc1)C(=O)O] 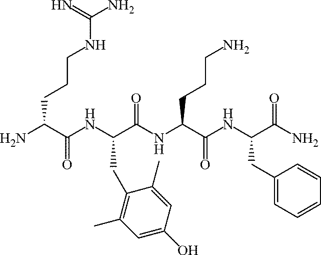 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(N)=O] 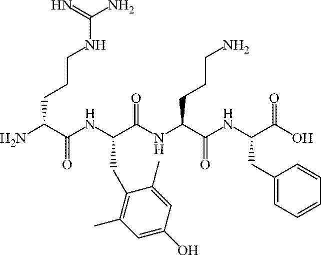 [SMILES: Cc1cc(O)cc(C)c1C[C@H](NC(=O)[C@H](N)CCCNC(=N)N)C(=O)N[C@@H](CCCN)C(=O)N[C@@H](Cc1ccccc1)C(=O)O] 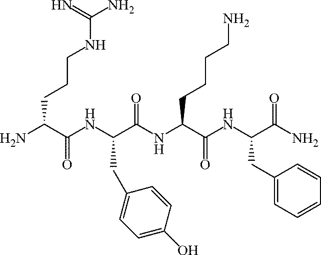 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(N)=O] 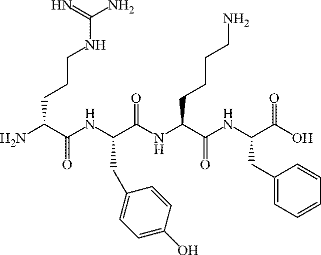 [SMILES: N=C(N)NCCC[C@@H](N)C(=O)N[C@@H](Cc1ccc(O)cc1)C(=O)N[C@@H](CCCCN)C(=O)N[C@@H](Cc1ccccc1)C(=O)O]
or a pharmaceutically acceptable salt, hydrate, solvate and/or tautomer thereof.

37. The use of any one of claims 34 to 36, wherein the medicament further comprises a drug known to increase or correct the production of dystrophin in the subject.

38. The use of claim 37, wherein the subject has been diagnosed as having DMD and the drug known to increase or correct the production of dystrophin is a phosphorodiamidate morpholino oligomer (PMO), such as Eteplirsen (Exondys 51®), Golodirsen (Vyondys 53™) or Casimersen (Amondys 45™), or a PPMO.

39. The use of any one of claims 34 to 38, wherein the muscular dystrophy is Duchene muscular dystrophy.

40. The use of any one of claims 34 to 38, wherein the muscular dystrophy is Becker muscular dystrophy.

41. The use of any one of claims 34 to 40, wherein the cardiomyopathy is hypertrophic cardiomyopathy.

42. The use of any one of claims 34 to 40, wherein the cardiomyopathy is dilated cardiomyopathy.

43. The use of any one of claims 34 to 40, wherein the cardiomyopathy is heart failure.

44. The use of any one of claims 34 to 40, wherein the cardiomyopathy is cardiac fibrosis.

45. The use of any one of claims 34 to 44, wherein the medicament increases ejection fraction of the heart of the subject as compared with the heart of an untreated control subject or control group that is not administered the composition.

46. The use of any one of claims 34 to 44, wherein the medicament increases shortening fraction of the heart of the subject as compared with the heart of an untreated control subject or control group that is not administered the composition.

47. The use of any one of claims 34 to 44, wherein the medicament increases stroke volume of the heart of the subject as compared with the heart of an untreated control subject or control group that is not administered the composition.

48. The use of any one of claims 34 to 44, wherein the medicament increases cardiac output of the heart of the subject as compared with the heart of an untreated control subject or control group that is not administered the composition.

49. The use of any one of claims 34 to 44, wherein the medicament prevents, inhibits, reduces, ameliorates, and/or delays the onset of cardiac fibrosis in the subject as compared with the heart of an untreated control subject or control group that is not administered the composition.

50. The use of any one of claims 34 to 49, wherein the medicament is a depot formulation.

51. The use of claim 50, wherein the depot formulation comprises the peptide of Formula A encapsulated or otherwise disposed in silica microparticles.

52. The use of claims 50 or 51, wherein the depot formulation is a sustained release depot formulation.

53. The use of claim 52, wherein the peptide of Formula A is released in an effective amount over days, weeks or months.

54. A composition comprising:
a) a peptide of formula A: 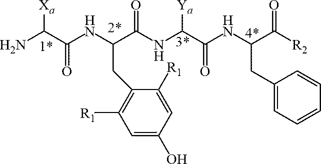
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 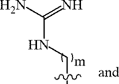 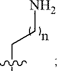
each m is 2, 3 or 4;
each n is independently 1, 2, or 3; and the absolute stereochemistry at each of stereocenters 1*, 2*, 3*, and 4* is independently D or L; and
b) a drug known to increase or correct the production of dystrophin in a subject.

55. The composition of claim 54, wherein the peptide is A-1 or A-2: 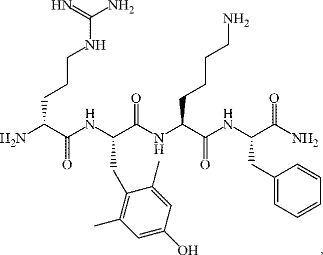 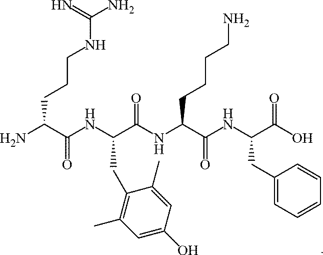
.

56. The composition of claims 54 or 55, wherein the drug known to increase or correct the production of dystrophin is a phosphorodiamidate morpholino oligomer (PMO), such as Eteplirsen (Exondys 51®), Golodirsen (Vyondys 53™) or Casimersen (Amondys 45™), or a PPMO.

57. The composition of any one of claims 54 to 56, wherein the composition is a medicament.

58. A method for treating cardiomyopathy or delaying the onset of cardiomyopathy in a mammalian subject suffering from muscular dystrophy, comprising administering to the subject a therapeutically effective amount of a composition of any one of claims 55 to 58.

59. The method of claim 58, wherein the composition is administered daily, weekly or monthly.

60. The method of claims 58 or 59, wherein the composition is administered intravenously.

61. The method of any one of claims 58 to 60, wherein the muscular dystrophy is Duchene muscular dystrophy or Becker muscular dystrophy.

62. A formulation comprising a peptide of formula A: 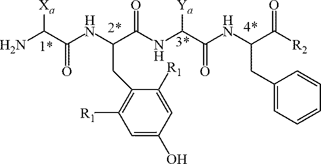
or a pharmaceutically acceptable salt, hydrate, solvate, and/or tautomer thereof, wherein, each R₁ is independently H or —CH₃; R₂ is —OH or —NH₂; Xₐ and Yₐ are each independently selected from 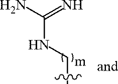 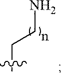
each m is 2, 3 or 4;
each n is independently 1, 2, or 3; and the absolute stereochemistry at each of stereocenters 1*, 2*, 3*, and 4* is independently D or L, wherein: (i) said peptide is encapsulated by, or disposed within, silica microparticles; and (ii) said silica microparticles are formulated for systemic delivery of the peptide to a subject over days, weeks or months to thereby deliver an effective dose to thereby treat the subject for one or more signs, symptoms, or risk factors of cardiomyopathy associated with MD, DMD, or BMD.
